(19) United States
(12) Patent Application Publication
(10) Pub. No.: US 2011/0143358 A1
(43) Pub. Date: Jun. 16, 2011

(54) COMPOSITIONS FOR USE IN IDENTIFICATION OF TICK-BORNE PATHOGENS

(75) Inventors: Rangarajan Sampath, San Diego, CA (US); David J. Ecker, Encinitas, CA (US); Mark W. Eshoo, Solana Beach, CA (US); Heather E. Matthews, Carlsbad, CA (US); Christopher D. Crowder, Carlsbad, CA (US); Lawrence B. Blyn, Mission Viejo, CA (US)

(73) Assignee: IBIS BIOSCIENCES, INC., Carlsbad, CA (US)

(21) Appl. No.: 12/995,429

(22) PCT Filed: May 29, 2009
(86) PCT No.: PCT/US09/45660

§ 371 Date: Feb. 28, 2011

Related U.S. Application Data
(60) Provisional application No. 61/057,566, filed on May 30, 2008.

(51) Int. Cl. C12Q 1/68; H01J 49/26

(52) U.S. Cl. 435/6.12; 250/281

(57) ABSTRACT

The present invention relates generally to the field of genetic identification and quantification of tick borne pathogens and provides methods, compositions and kits useful for this purpose when combined with molecular mass or base composition analysis.

### CROSS REFERENCE TO RELATED APPLICATIONS

This application claims the benefit of U.S. Provisional Patent Application Ser. No. 61/057,566, filed May 30, 2008, which is herein incorporated by reference in its entirety.

### FIELD OF THE INVENTION

The present invention relates generally to the field of genetic identification and quantification of tick borne pathogens and provides methods, compositions and kits useful for this purpose when combined with molecular mass or base composition analysis.

### BACKGROUND OF THE INVENTION

Tick-borne pathogens represent a large portion of vector-borne disease causing organisms. Performing risk-assessment and determining the prevalence of the pathogens previously required a specific test for each distinct pathogen resulting in costly and time-consuming processes, which could not always detect novel species or strains.

What are needed are improved methods and compositions for identifying and characterizing tick-borne pathogens.

### SUMMARY OF THE INVENTION

Provided herein are, inter alia, compositions, kits, and methods of identifying tick-borne pathogens and endosymbionts. Also provided are oligonucleotide primers, compositions and kits containing oligonucleotide primers that upon amplification, produce amplicons whose molecular masses provide the means to identify, for example, tick-borne pathogens (e.g., bacteria) and endosymbionts at the species or sub-species level. These compositions and methods permit comprehensive identification and monitoring of tick-borne pathogens, as well as identification and monitoring of ticks. In certain embodiments, related systems of use in the detection and identification of tick-borne pathogens and endosymbionts are also provided.

In some embodiments, the invention provides primers, and compositions comprising pairs of primers; kits containing the same; and methods for their use in the identification of tick-borne pathogens and endosymbionts. The primers are typically configured to produce pathogen or endosymbiont (e.g., bacteria) bioagent-identifying nucleic acid amplicons i.e. amplification products. The amplicons are typically generated from regions of nucleic acid encoding unique (e.g., variable) regions of the tick-borne pathogens and endosymbionts. Compositions comprising pairs of primers and the kits containing the same are generally configured to provide species and sub-species characterization of, for example, Spirochetes, alphaproteobacteria, Francisella, Coxiella Burnetii, Flaviviruses, Borrelia, Rickettsia, Anaplasma, Sprioplasma, Ehrlichia, Wolbachia, and Bartonella.

In another aspect, the invention provides a composition comprising at least one purified oligonucleotide primer pair that comprises forward and reverse primers, wherein the primer pair comprises nucleic acid sequences that are substantially complementary to nucleic acid sequences of two or more different tick-borne pathogens and/or or endosymbiont bioagent, wherein the primer pair is configured to produce amplicons comprising different base compositions that correspond to (i.e., match, identify, or otherwise correlate with) the two or more different bioagents. In some embodiments, the primer pair is configured to hybridize with conserved regions of two or more different bioagents and flank variable regions of the two or more different bioagents. In further embodiments, the forward and reverse primers are about 15 to 35 nucleobases in length, and the forward primer comprises at least 70%, at least 80%, at least 90%, at least 95%, or at least 100% sequence identity with a sequence of SEQ ID NOS: 1-18, and the reverse primer comprises at least 70% sequence identity with a sequence of SEQ ID NOS: 19-36. In some embodiments, the forward and reverse primers are about 15 to 35 nucleobases in length, and the forward primer comprises at least 70%, at least 80%, at least 90%, at least 95%, or at least 100% sequence identity with the sequence of SEQ ID NO:1, and the reverse primer comprises at least 70%, at least 80%, at least 90%, at least 95%, or at least 100% sequence identity with the sequence of SEQ ID NO: 19; the forward primer comprises at least 70%, at least 80%, at least 90%, at least 95%, or at least 100% sequence identity with the sequence of SEQ ID NO: 2, and the reverse primer comprises at least 70%, at least 80%, at least 90%, at least 95%, or at least 100% sequence identity with the sequence of SEQ ID NO: 20; the forward primer comprises at least 70%, at least 80%, at least 90%, at least 95%, or at least 100% sequence identity with the sequence of SEQ ID NO: 3, and the reverse primer comprises at least 70%, at least 80%, at least 90%, at least 95%, or at least 100% sequence identity with the sequence of SEQ ID NO: 21; the forward primer comprises at least 70%, at least 80%, at least 90%, at least 95%, or at least 100% sequence identity with the sequence of SEQ ID NO: 4, and the reverse primer comprises at least 70%, at least 80%, at least 90%, at least 95%, or at least 100% sequence identity with the sequence of SEQ ID NO: 22; the forward primer comprises at least 70%, at least 80%, at least 90%, at least 95%, or at least 100% sequence identity with the sequence of SEQ ID NO: 5, and the reverse primer comprises at least 70%, at least 80%, at least 90%, at least 95%, or at least 100% sequence identity with the sequence of SEQ ID NO: 23; the forward primer comprises at least 70%, at least 80%, at least 90%, at least 95%, or at least 100% sequence identity with the sequence of SEQ ID NO: 6, and the reverse primer comprises at least 70%, at least 80%, at least 90%, at least 95%, or at least 100% sequence identity with the sequence of SEQ ID NO: 24; the forward primer comprises at least 70%, at least 80%, at least 90%, at least 95%, or at least 100% sequence identity with the sequence of SEQ ID NO: 7, and the reverse primer comprises at least 70%, at least 80%, at least 90%, at least 95%, or at least 100% sequence identity with the sequence of SEQ ID NO: 25; the forward primer comprises at least 70%, at least 80%, at least 90%, at least 95%, or at least 100% sequence identity with the sequence of SEQ ID NO: 8, and the reverse primer comprises at least 70%, at least 80%, at least 90%, at least 95%, or at least 100% sequence identity with the sequence of SEQ ID NO: 26; the forward primer comprises at least 70%, at least 80%, at least 90%, at least 95%, or at least 100% sequence identity with the sequence of SEQ ID NO: 9, and the reverse primer comprises at least 70%, at least 80%, at least 90%, at least 95%, or at least 100% sequence identity with the sequence of SEQ ID NO: 27; the forward primer comprises at least 70%, at least 80%, at least 90%, at least 95%, or at least 100% sequence identity with the sequence of SEQ ID NO: 10, and the reverse primer comprises at least 70%, at least 80%, at least 90%, at least 95%, or at least 100% sequence identity with the sequence of SEQ ID NO: 28; the forward primer comprises at least 70%, at least 80%, at least 90%, at least 95%, or at least 100% sequence identity with the sequence of SEQ ID NO: 11, and the reverse primer comprises at least 70%, at least 80%, at least 90%, at least 95%, or at least 100% sequence identity with the sequence of SEQ ID NO: 29; the forward primer comprises at least 70%, at least 80%, at least 90%, at least 95%, or at least 100% sequence identity with the sequence of SEQ ID NO: 12, and the reverse primer comprises at least 70%, at least 80%, at least 90%, at least 95%, or at least 100% sequence identity with the sequence of SEQ ID NO: 30; the forward primer comprises at least 70%, at least 80%, at least 90%, at least 95%, or at least 100% sequence identity with the sequence of SEQ ID NO: 13, and the reverse primer comprises at least 70%, at least 80%, at least 90%, at least 95%, or at least 100% sequence identity with the sequence of SEQ ID NO: 31; the forward primer comprises at least 70%, at least 80%, at least 90%, at least 95%, or at least 100% sequence identity with the sequence of SEQ ID NO: 14, and the reverse primer comprises at least 70%, at least 80%, at least 90%, at least 95%, or at least 100% sequence identity with the sequence of SEQ ID NO: 32; the forward primer comprises at least 70%, at least 80%, at least 90%, at least 95%, or at least 100% sequence identity with the sequence of SEQ ID NO: 15, and the reverse primer comprises at least 70%, at least 80%, at least 90%, at least 95%, or at least 100% sequence identity with the sequence of SEQ ID NO: 33; the forward primer comprises at least 70%, at least 80%, at least 90%, at least 95%, or at least 100% sequence identity with the sequence of SEQ ID NO: 16, and the reverse primer comprises at least 70%, at least 80%, at least 90%, at least 95%, or at least 100% sequence identity with the sequence of SEQ ID NO: 34; the forward primer comprises at least 70%, at least 80%, at least 90%, at least 95%, or at least 100% sequence identity with the sequence of SEQ ID NO: 17, and the reverse primer comprises at least 70%, at least 80%, at least 90%, at least 95%, or at least 100% sequence identity with the sequence of SEQ ID NO: 35; and/or, the forward primer comprises at least 70%, at least 80%, at least 90%, at least 95%, or at least 100% sequence identity with the sequence of SEQ ID NO: 18, and the reverse primer comprises at least 70%, at least 80%, at least 90%, at least 95%, or at least 100% sequence identity with the sequence of SEQ ID NO: 36.

In some embodiments, the different base compositions identify two or more different bioagents at the genus, species, or sub-species levels. In other embodiments, the two or more amplicons are 45 to 200 nucleobases in length. In still other embodiments, the different bioagents are selected from a Spirochete, an alphaproteobacteria, a Francisella sp., Coxiella burnetii, a Flavivirus, Borrelia miyamotoi, Borrelia burgdorferi type 1, Borrelia burgdorferi type 2, Borrelia lonestari, Spotted Fever Group Rickettsia variant 1, Spotted Fever Group Rickettsia variant 2, Spotted Fever Group Rickettsia variant 3, Rickettsia amblyommii, Anaplasma phagocytophilum, Spiroplasma ixodetis, Ehrlichia variant 1, Wolbachia variant 1, Bartonella variant 1, Bartonella variant 2, Bartonella variant 3, Francisella genus or combinations thereof. In further embodiments, the primer pair is configured to hybridize with one or more nucleic acid sequences selected from, e.g., flagellin, rplB, gyrB, hbb, rnpA, gltA, rpoB, asd, gale, icd, IS1111A, S. kunkeii TraE-like gene, 16S rRNA gene, 23S rRNA gene and RdRp nucleic acids.

In some embodiments, a non-templated T residue on the 5′-end of the forward and/or reverse primer is removed. In still other embodiments, the forward and/or reverse primer further comprises a non-templated T residue on the 5′-end. In additional embodiments, the forward and/or reverse primer comprises at least one molecular mass modifying tag. In some embodiments, the forward and/or reverse primer comprises at least one modified nucleobase. In further embodiments, the modified nucleobase is 5-propynyluracil or 5-propynylcytosine. In other embodiments, the modified nucleobase is a mass modified nucleobase. In still other embodiments, the mass modified nucleobase is 5-Iodo-C. In additional embodiments, the modified nucleobase is a universal nucleobase. In some embodiments, the universal nucleobase is inosine. In certain embodiments, kits comprise the compositions described herein.

In another aspect, the invention provides a kit comprising at least one purified oligonucleotide primer pair that comprises forward and reverse primers that are about 20 to 35 nucleobases in length, and wherein the forward primer comprises at least 70%, at least 80%, at least 90%, at least 95%, or at least 100% sequence identity with a sequence selected from the group consisting of SEQ ID NOS: 1-18, and the reverse primer comprises at least 70% sequence identity with a sequence selected from the group consisting of SEQ ID NOS: 19-36.

In another aspect, the invention provides a method of determining a presence of a tick-borne pathogen and/or or endosymbiont in at least one sample. The method includes (a) amplifying one or more segments of at least one nucleic acid from the sample using at least one purified oligonucleotide primer pair that comprises forward and reverse primers that are about 20 to 35 nucleobases in length, and wherein the forward primer comprises at least 70%, at least 80%, at least 90%, at least 95%, or at least 100% sequence identity with a sequence selected from the group consisting of SEQ ID NOs: 1-18, and the reverse primer comprises at least 70% sequence identity with a sequence selected from the group consisting of SEQ ID NOs: 19-36 to produce at least one amplification product. In addition, the method also includes (b) detecting the amplification product, thereby determining the presence of the tick-borne pathogen and/or or endosymbiont in the sample. In some embodiments, (a) comprises amplifying the one or more segments of the at least one nucleic acid from at least two samples obtained from different geographical locations to produce at least two amplification products, and (b) comprises detecting the amplification products, thereby tracking an epidemic spread of the tick-borne pathogen and/or endosymbiont. Optionally, (b) comprises determining an amount of the tick-borne pathogen and/or endosymbiont in the sample (e.g., determining a bacterial titer or the like). Typically, (b) comprises detecting a molecular mass of the amplification product. In some embodiments, (b) comprises determining a base composition of the amplification product in which the base composition identifies the number of A residues, C residues, T residues, G residues, U residues, analogs thereof and/or mass tag residues thereof in the amplification product, whereby the base composition indicates the presence of tick-borne pathogens and/or or endosymbionts in the sample or identifies the tick-borne pathogens and/or or endosymbiont in the sample. In certain embodiments, the method includes comparing the base composition of the amplification product to calculated or measured base compositions of amplification products of one or more known tick-borne pathogens and/or or endosymbionts present in a database with the proviso that sequencing of the amplification product is not used to indicate the presence of or to identify the tick-borne pathogen and/or or endosymbiont in which a match between the determined base composition and the calculated or measured base composition in the database indicates the presence of or identifies the tick-borne pathogen and/or endosymbiont.

In another aspect, the invention provides a method of identifying one or more tick-borne pathogen and/or endosymbiont in a sample. The method includes (a) amplifying two or more segments of a nucleic acid from the one or more tick-borne pathogen and/or endosymbiont bioagents in the sample with two or more oligonucleotide primer pairs to obtain two or more amplification products; (b) determining two or more molecular masses and/or base compositions of the two or more amplification products; and (c) comparing the two or more molecular masses and/or the base compositions of the two or more amplification products with known molecular masses and/or known base compositions of amplification products of known tick-borne pathogen and/or endosymbiont bioagents produced with the two or more primer pairs to identify the one or more tick-borne pathogen and/or endosymbiont bioagents in the sample. In some embodiments, the method includes identifying the one or more tick-borne pathogen and/or endosymbiont bioagents in the sample using three, four, five, six, seven, eight or more primer pairs. Optionally, the two or more segments of the nucleic acid are amplified from a single gene, or the two or more segments of the nucleic acid are amplified from different genes. In some embodiments, the one or more tick-borne pathogen and/or endosymbiont bioagents in the sample cannot be identified using a single primer pair of the two or more primer pairs. Typically, the method includes obtaining the two or more molecular masses of the two or more amplification products via mass spectrometry. In certain embodiments, the one or more tick-borne pathogen and/or endosymbiont in the sample cannot be identified using a single primer pair of the two or more primer pairs.

In some embodiments, the tick-borne pathogens and/or or endosymbionts are selected from: a Spirochete, an alphaproteobacteria, a Francisella sp., Coxiella burnetii, a Flavivirus, Borrelia miyamotoi, Borrelia burgdorferi type 1, Borrelia burgdorferi type 2, Borrelia lonestari, Spotted Fever Group Rickettsia variant 1, Spotted Fever Group Rickettsia variant 2, Spotted Fever Group Rickettsia variant 3, Rickettsia amblyommii, Anaplasma phagocytophilum, Spiroplasma ixodetis, Ehrlichia variant 1, Wolbachia variant 1, Bartonella variant 1, Bartonella variant 2, Bartonella variant 3, Francisella genus or combinations thereof. In some embodiments, the primer pairs are selected from the group of primer pair sequences of: SEQ ID NOS: 1:19, 2:20, 3:21, 4:22, 5:23, 6:24, 7:25, 8:26, 9:27, 10:28, 11:29, 12:30, 13:31, 14:32, 15:33, 16:34, 17:35, or 18:36

Typically, the determining the one or more molecular masses and/or base compositions is conducted without sequencing the amplification products. In some embodiments, the tick-borne pathogen and/or or endosymbiont bioagents in a sample are identified by comparing three or more molecular masses and/or base compositions of three or more amplification products with a database of known molecular masses and/or known base compositions of amplification products of known tick-borne pathogen and/or or endosymbiont bioagents produced with the three or more primer pairs. In certain embodiments, the method includes calculating base compositions from molecular masses of amplification products.

In some embodiments, members of the primer pairs hybridize to conserved regions of nucleic acid that flank a variable region. Typically, the variable region varies between at least two of the tick-borne pathogen and/or or endosymbiont bioagents. In some embodiments, the variable region uniquely varies between at least five of the tick-borne pathogen and/or or endosymbiont bioagents.

In certain embodiments, the amplification products obtained in (a) comprise major classification and subgroup identifying amplification products. In some embodiments, the method includes comparing the molecular masses and/or the base compositions of the amplification products to calculated or measured molecular masses or base compositions of amplification products of known tick-borne pathogen and/or or endosymbiont bioagents in a database comprising genus specific amplification products, species specific amplification products, strain specific amplification products or nucleotide polymorphism specific amplification products produced with the oligonucleotide primer pairs in which one or more matches between the amplification products and one or more entries in the database identifies one or more tick-borne pathogen and/or or endosymbiont bioagents, classifies a major classification of one or more tick-borne pathogen and/or or endosymbiont bioagents, and/or differentiates between subgroups of known and unknown tick-borne pathogen and/or or endosymbiont bioagents in the sample. In some of these embodiments, the major classification of one or more tick-borne pathogen and/or or endosymbiont bioagents comprises a genus or species classification of one or more tick-borne pathogen and/or or endosymbiont bioagents. In some of these embodiments, subgroups of known and unknown tick-borne pathogen and/or or endosymbiont bioagents comprise family, strain and nucleotide variations of the one or more tick-borne pathogen and/or or endosymbiont bioagents.

In another aspect, the invention provides a system that includes (a) a mass spectrometer configured to detect one or more molecular masses of amplicons produced using at least one purified oligonucleotide primer pair that comprises forward and reverse primers in which the primer pair comprises nucleic acid sequences that are substantially complementary to nucleic acid sequences of one or more tick-borne pathogen and/or or endosymbiont bioagents. The system also includes (b) a controller operably connected to the mass spectrometer, the controller configured to correlate molecular masses of amplicons with one or more tick-borne pathogen and/or or endosymbiont bioagent identities (e.g., at genus, species, and/or sub-species levels). In some embodiments, the forward and reverse primers are about 15 to 35 nucleobases in length, and wherein the forward primer comprises at least 70%, at least 80%, at least 90%, at least 95%, or at least 100% sequence identity with a sequence selected from SEQ ID NOS: 1-18, and the reverse primer comprises at least 70% sequence identity with a sequence selected from SEQ ID NOS: 19-36. Typically, the controller is configured to determine (e.g., calculate, etc.) base compositions of amplicons from molecular masses of amplicons, which base compositions correspond to (i.e., elucidate or otherwise correlate with) one or more tick-borne pathogen and/or or endosymbiont bioagent identities. In some embodiments, the controller comprises or is operably connected to a database of known molecular masses and/or known base compositions of amplicons of known tick-borne pathogen and/or endosymbiont bioagents produced with the primer pair.

In certain aspects, methods for identification of tick-borne pathogen and/or endosymbiont are provided. Nucleic acid from the members of the tick-borne pathogen and/or endosymbiont is amplified using the primers described herein to obtain an amplicon. The molecular mass of the amplicon is measured using mass spectrometry. In some embodiments, a base composition of the amplicon is calculated from the molecular mass. As used herein, the term “base composition” refers to the number of each residue comprising an amplicon, without consideration for the linear arrangement of these residues in the strand(s) of the amplicon, wherein the base composition identifies the number of A residues, C residues, T residues, G residues, U residues, analogs thereof and/or mass tag residues thereof in the amplification product. The molecular mass or base composition is typically compared with a plurality of molecular masses or base compositions in a database of known tick-borne pathogen and/or endosymbiont identifying amplicons, wherein a match between the molecular mass or base composition and a member of the plurality of molecular masses or base compositions identifies the tick-borne pathogen and/or endosymbiont.

In some embodiments, methods of detecting the presence or absence of a tick-borne pathogen and/or or endosymbiont in a sample are provided. Nucleic acid from the sample is amplified using the composition described above to obtain an amplicon. The molecular mass of this amplicon is determined by mass spectrometry. A base composition of the amplicon is determined from the molecular mass without sequencing the amplicon. The molecular mass or base composition of the amplicon is compared with known molecular masses or base compositions in a database of one or more known tick-borne pathogen and/or or endosymbiont identifying amplicons, wherein a match between the molecular mass or base composition of the amplicon and the molecular mass or base composition of one or more known tick-borne pathogen and/or or endosymbiont identifying amplicons indicates the presence of the tick-borne pathogen and/or or endosymbiont in the sample.

In certain embodiments, methods for determination of the quantity of an unknown tick-borne pathogen and/or or endosymbiont in a sample are provided. The sample is contacted with the composition described herein and a known quantity of a calibration polynucleotide. Nucleic acid from the unknown tick-borne pathogen and/or or endosymbiont in the sample is concurrently amplified with the composition described above and nucleic acid from the calibration polynucleotide in the sample is concurrently amplified with the composition described above to obtain a first amplicon comprising a tick-borne pathogen and/or or endosymbiont identifying amplicon and a second amplicon comprising a calibration amplicon. The molecular mass and abundance for the tick-borne pathogen and/or or endosymbiont identifying amplicon and the calibration amplicon is determined by mass spectrometry. The tick-borne pathogen and/or or endosymbiont identifying amplicon is distinguished from the calibration amplicon based on molecular mass, wherein comparison of tick-borne pathogen and/or or endosymbiont identifying amplicon abundance and calibration amplicon abundance indicates the quantity of tick-borne pathogen and/or or endosymbiont in the sample. The base composition of the tick-borne pathogen and/or or endosymbiont identifying amplicon is determined.

In some embodiments, a method of identifying one or more tick-borne pathogen and/or or endosymbiont bioagents in a sample is provided, comprising the steps of (a) amplifying two or more segments of a nucleic acid from the one or more of tick-borne pathogen and/or or endosymbiont bioagents in the sample with two or more primer pairs to obtain two or more amplification products, wherein each of the primer pairs hybridizes to conserved regions of the nucleic acid that flank a variable region; (b) determining two or more molecular masses of the two or more amplification products; and (c) comparing the two or more molecular masses with a database containing known molecular masses of known tick-borne pathogen and/or or endosymbiont bioagents produced with the two or more primer pairs to identify one or more tick-borne pathogen and/or or endosymbiont bioagents in the sample. In some embodiments, the two or more primer pairs comprise two or more purified oligonucleotide primer pairs wherein the forward and reverse members of the two or more primer pairs are 20 to 35 nucleobases in length, and wherein the forward members comprises at least 70%, at least 80%, at least 90%, at least 95%, or at least 100% sequence identity with a sequence selected from the group consisting of SEQ ID NOS: 1-18, and the reverse members comprises at least 70% sequence identity with a sequence selected from the group consisting of SEQ ID NOS: 19-36 to obtain an amplification product. In other embodiments, the determining of two or more molecular masses of the two or more amplification products is conducted without sequencing. In further embodiments, the variable region varies between at least two or the tick-borne pathogen and/or or endosymbiont bioagents. In still further embodiments, the variable region uniquely varies between at least five of the tick-borne pathogen and/or or endosymbiont bioagents. In certain embodiments, the molecular masses of the two or more amplification products are obtained via mass spectrometry. In some embodiments, the one or more tick-borne pathogen and/or or endosymbiont bioagents in the sample cannot be identified using a single primer pair of the two or more primer pairs. In additional embodiments, the one or more tick-borne pathogen and/or or endosymbiont bioagents in a sample are identified by comparing three or more molecular masses to a database of bioagents produced with three or more primer pairs. In other embodiments, the two or more segments of a nucleic acid are amplified from a single gene. In still other embodiments, the two or more segments of a nucleic acid are amplified from different genes.

In some embodiments, a method of identifying one or more tick-borne pathogen and/or or endosymbiont bioagents in a sample is provided, comprising (a) providing two or more oligonucleotide primer pairs wherein a forward member of the pair of primers hybridizes to a first conserved sequence of nucleic acid from the one or more tick-borne pathogen and/or or endosymbiont bioagents and a reverse member of the pair of primers hybridizes to a second conserved sequence of nucleic acid from the one or more tick-borne pathogen and/or or endosymbiont bioagents wherein the first and second conserved sequences flank a variable nucleic acid sequence that varies among different tick-borne pathogen and/or or endosymbiont bioagents; (b) providing nucleic acid from the sample; (c) amplifying two or more segments of the nucleic acid from the one or more tick-borne pathogen and/or or endosymbiont bioagents in the sample with the two or more oligonucleotide primer pairs to obtain two or more major classification and subgroup identifying amplification products; (d) determining molecular masses by mass spectrometry or base compositions by mass spectrometry of the two or more amplification products; and (e) comparing the molecular masses or the base compositions of the two or more amplification products to calculated or measured molecular masses or base compositions of amplification products of known tick-borne pathogen and/or or endosymbiont bioagents in a database comprising genus specific amplification products, species specific amplification products, strain specific amplification products or nucleotide polymorphism specific amplification products produced with the two or more oligonucleotide primer pairs, wherein a match between the two or more amplification products and one or more entries in the database identifies the one or more tick-borne pathogen and/or or endosymbiont bioagents, and wherein a first match classifies a major classification of the one or more tick-borne pathogen and/or or endosymbiont bioagents, and a second match differentiates between subgroups of known and unknown tick-borne pathogen and/or or endosymbiont bioagents in the sample. In some embodiments, the major classification of the one or more tick-borne pathogen and/or or endosymbiont bioagents comprises genus or species classification of the one or more tick-borne pathogen and/or or endosymbiont bioagents. In other embodiments, the subgroups of known and unknown tick-borne pathogen and/or or endosymbiont bioagents comprise family, strain and nucleotide variations of the one or more tick-borne pathogen and/or or endosymbiont bioagents. In still other embodiments, the family of the one or more tick-borne pathogen and/or or endosymbiont bioagents comprises the tick-borne pathogen and/or or endosymbiont family. In some embodiments, the forward primer member comprises at least 70%, at least 80%, at least 90%, at least 95%, or at least 100% sequence identity with a sequence selected from the group consisting of SEQ ID NOs: 1-18, and the reverse primer member comprises at least 70%, at least 80%, at least 90%, at least 95%, or at least 100% sequence identity with a sequence selected from the group consisting of SEQ ID NOs: 19-36. In additional embodiments, either or both of the members of the pair of primers comprises at least one modified nucleobase. In further embodiments, the modified nucleobase is a mass modified nucleobase or is a universal nucleobase. In still further embodiments, the universal nucleobase is inosine. In other embodiments, the mass modified nucleobase is 5-Iodo-C. In some embodiments, a non-templated T residue is added to the 5′-end on either or both of the primer pair members. In other embodiments, either or both of the forward and the reverse primer pair members further comprises a non-templated T residue on the 5′-end. In certain embodiments, the determining of the base compositions of the two or more amplification products is conducted without sequencing. In some embodiments, the variable sequence uniquely varies between at least five of the bioagents. In other embodiments, the base compositions of the two or more amplification products are calculated from molecular masses of the two or more amplification products. In still other embodiments, the one or more bioagents in the sample cannot be identified using a single primer pair of the two or more primer pairs. In further embodiments, the one or more bioagents in a sample are identified by comparing three or more base compositions to a database of bioagents produced with three or more primer pairs. In other embodiments, the two or more segments of the nucleic acid are amplified from a single gene. In still other embodiments, the two or more segments of the nucleic acid are amplified from different genes.

In some embodiments, a composition comprising a combination of at least three purified oligonucleotide primer pairs is provided, wherein the primer pairs hybridize to two or more genes selected from the group of FLAGELLIN, RPLB, GYRB, HBB, RNPA, GLTA, RPOB, ASD, GALE, ICD, IS1111A, S. KUNKEII TRAE-LIKE GENE, 165 RRNA GENE, 23S RRNA GENE AND RDRP, wherein the primer pairs hybridize with conserved regions of the genes and flank variable regions of the genes to generate two or more amplicons from the two or more genes, wherein the two or more amplicons are configured to generate two or more molecular mass measurements using mass spectrometry, and wherein the two or more amplicons are configured to generate two or more base compositions from the molecular mass measurements that correspond to two or more unknown tick-borne pathogen and/or endosymbiont bioagents. In some embodiments, the primer pairs individually bind to one or more genes from the group of FLAGELLIN, RPLB, GYRB, HBB, RNPA, GLTA, RPOB, ASD, GALE, ICD, IS1111A, S. KUNKEII TRAE-LIKE GENE, 16S RRNA GENE, 23S RRNA GENE AND RDRP genes.

In some embodiments, a method of tracking the epidemic spread of pathogen and/or endosymbiont is provided, comprising (a) providing a one or more samples containing the tick-borne pathogen and/or endosymbiont from a plurality of locations; (b) providing pathogen and/or endosymbiont RNA and/or DNA from the one or more samples; (c) amplifying the DNA with a purified oligonucleotide primer pair wherein the forward and reverse members of the primer pair are 20 to 35 nucleobases in length, and wherein the forward primer comprises at least 70%, at least 80%, at least 90%, at least 95%, or at least 100% sequence identity with a sequence selected from the group consisting of SEQ ID NOS: 1-18, and the reverse primer comprises at least 70% sequence identity with a sequence selected from the group consisting of SEQ ID NOS: 19-36 to produce an amplification product; and (d) identifying the tick-borne pathogen and/or endosymbiont in a subset of the one or more samples, wherein the amplification product identifies the tick-borne pathogen and/or endosymbiont and wherein the corresponding locations of the members of the subset indicate the epidemic spread of the tick-borne pathogen and/or endosymbiont to the corresponding locations. In some embodiments the method further comprises contacting the DNA with at least one primer pair comprising a forward member and a reverse member comprising oligonucleotide primers which hybridize to flanking sequences of the DNA, wherein the flanking sequences flank a variable DNA sequence corresponding to a variable RNA sequence of the tick-borne pathogen and/or endosymbiont. In other embodiments, the method further comprises determining the base composition of the amplification product by mass spectrometry, wherein the base composition identifies the number of A residues, C residues, T residues, G residues, U residues, analogs thereof and mass tag residues thereof in the amplification product. In further embodiments, the method further comprises comparing the base composition of the amplification product to calculated or measured base compositions of amplification products of one or more known tick-borne pathogen and/or endosymbiont present in a database with the proviso that sequencing of the amplification product is not used to identify the tick-borne pathogen and/or endosymbiont, wherein a match between the determined base composition and the calculated or measured base composition in the database identifies the in the two or more samples. In certain embodiments the mass spectrometry comprises ESI-TOF mass spectrometry.

In some embodiments, a method for simultaneous determination of the identity and quantity of a tick-borne pathogen and/or or endosymbiont in a sample is provided, comprising (a) contacting the sample with a pair of oligonucleotide primers and a known quantity of a calibration polynucleotide comprising a calibration polynucleotide sequence; (b) simultaneously amplifying the DNA from at least one tick-borne pathogen and/or or endosymbiont with the pair of oligonucleotide primers and amplifying nucleic acid from the calibration polynucleotide in the sample with the pair of oligonucleotide primers to obtain at least one tick-borne pathogen and/or or endosymbiont identifying amplification product and at least one calibration polynucleotide amplification product; (c) subjecting the sample to molecular mass analysis using a mass spectrometer wherein the result of the molecular mass analysis comprises molecular mass and abundance data for the tick-borne pathogen and/or or endosymbiont identifying amplification product and the calibration polynucleotide amplification product; and (d) distinguishing the tick-borne pathogen and/or or endosymbiont identifying amplification product from the calibration polynucleotide amplification product by molecular mass analysis wherein the molecular mass of the tick-borne pathogen and/or or endosymbiont identifying amplification product identifies at least one tick-borne pathogen and/or or endosymbiont in the sample, and comparison of the abundance of the tick-borne pathogen and/or or endosymbiont identifying amplification product and the calibration polynucleotide amplification product indicates the quantity of tick-borne pathogen and/or or endosymbiont in the sample. In some embodiments, the pair of oligonucleotide primers hybridize with a DNA sequence corresponding to a RNA sequence of at least three tick-borne pathogen and/or or endosymbiont family members and flank variable regions that vary between at least three tick-borne pathogen and/or or endosymbiont family members. In other embodiments, the calibration polynucleotide sequence comprises the sequence of a standard sequence of a tick-borne pathogen and/or or endosymbiont identifying amplification product further comprising the deletion of 2-8 consecutive nucleotide residues of the standard sequence in the calibration polynucleotide sequence. In still other embodiments, the calibration polynucleotide sequence comprises the sequence of a standard sequence of a tick-borne pathogen and/or or endosymbiont identifying amplification product further comprising the insertion of 2-8 consecutive nucleotide residues in the standard sequence in the calibration polynucleotide sequence. In additional embodiments, the calibration polynucleotide sequence comprises at least 80%, at least 90%, or at least 95% sequence identity with a standard sequence of a tick-borne pathogen and/or or endosymbiont identifying amplification product. In certain embodiments, the calibration polynucleotide resides on a plasmid. In other embodiments, the molecular mass analysis comprises ESI-TOF molecular mass analysis.

In some embodiments, a multiplex polymerase chain reaction method for identifying a tick-borne pathogen and/or or endosymbiont is provided comprising (a) providing a sample suspected of comprising one or more tick-borne pathogen and/or or endosymbionts; (b) providing DNA or RNA from the sample wherein the RNA or DNA comprises sequences encoding genes selected from flagellin, rplB, gyrB, hbb, rnpA, gltA, rpoB, asd, gale, icd, IS1111A, S. kunkeii TraE-like gene, 16S rRNA gene, 23S rRNA gene and RdRp nucleic acids genes; (c) amplifying the DNA to produce at least one amplification product using two or more oligonucleotide primer pairs; (d) determining the base composition of the at least one amplification product by mass spectrometry, wherein the base composition identifies the number of A residues, C residues, T residues, G residues, U residues, analogs thereof and mass tag residues thereof in the amplification product; and (e) comparing the base composition of the amplification product to calculated or measured base compositions of amplification products of one or more known tick-borne pathogen and/or or endosymbiont in a database with the proviso that sequencing of the amplification product is not used to identify the tick-borne pathogen and/or or endosymbiont, wherein a match between the determined base composition and the calculated or measured base composition in the database identifies the genus, species or strain of the one or more tick-borne pathogen and/or or endosymbiont family members in the sample. In some embodiments, at least one forward member of the two or more primer pairs comprises at least 70%, at least 80%, at least 90%, at least 95%, or at least 100% sequence identity with a sequence selected from the group consisting of SEQ ID NOs: 1-18, and at least one reverse member of the two or more primer pairs comprises at least 70% sequence identity with a sequence selected from the group consisting of SEQ ID NOs: 19-36. In certain embodiments, the amplifying is carried out in a single reaction vessel. In other embodiments, the amplifying is carried out in one or more primer pair specific reaction vessels. In still other embodiments, the one or more tick-borne pathogen and/or or endosymbiont family members are identified in the sample, the identified family members comprising one or more of a Spirochete, an alphaproteobacteria, a Francisella sp., Coxiella burnetii, a Flavivirus, Borrelia miyamotoi, Borrelia burgdorferi type 1, Borrelia burgdorferi type 2, Borrelia lonestari, Spotted Fever Group Rickettsia variant 1, Spotted Fever Group Rickettsia variant 2, Spotted Fever Group Rickettsia variant 3, Rickettsia amblyommii, Anaplasma phagocytophilum, Spiroplasma ixodetis, Ehrlichia variant 1, Wolbachia variant 1, Bartonella variant 1, Bartonella variant 2, Bartonella variant 3, Francisella genus or combinations thereof. In some embodiments, the mass spectrometry comprises ESI-TOF mass spectrometry.

### BRIEF DESCRIPTION OF THE DRAWINGS

The foregoing summary and detailed description is better understood when read in conjunction with the accompanying drawings which are included by way of example and not by way of limitation.

FIG. 1 shows a schematic of a tick-borne pathogen assay used in embodiments of the present invention.

FIG. 2 shows a schematic of PCR/ESI-MS methods used in embodiments of the present invention.

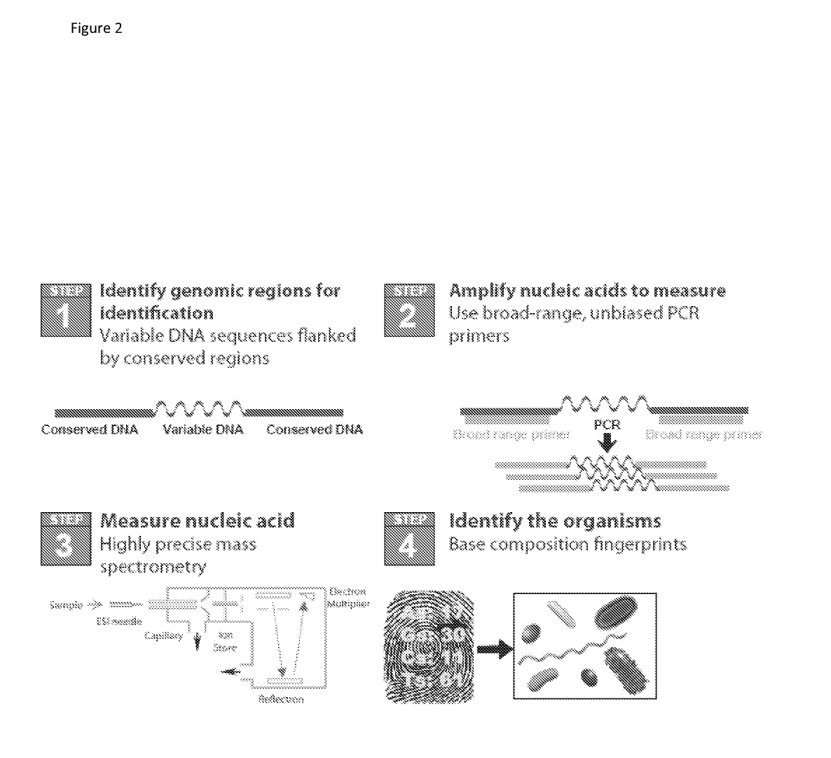

FIG. 3 shows a schematic of exemplary methods of total nucleic extraction from ticks.

FIG. 4 shows tick extractions of adult Dermacentor variabilis. Total nucleic acid (A), DNA only (B), and RNA only (C). Extract was treated with RNase (B) or DNase (C) to remove RNA and DNA respectively.

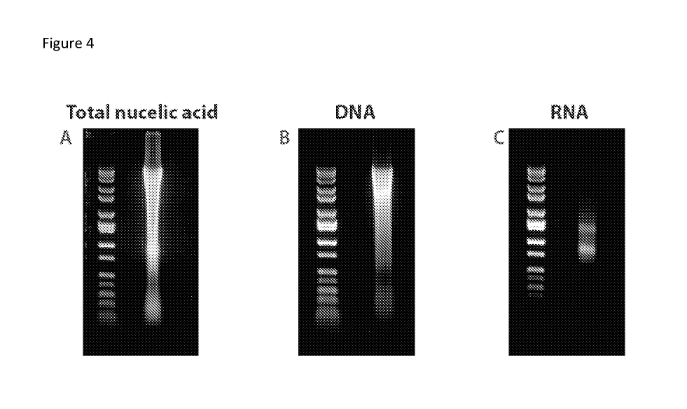

### DETAILED DESCRIPTION OF EMBODIMENTS

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. In describing and claiming the present invention, the following terminology and grammatical variants will be used in accordance with the definitions set forth below.

As used herein, the term “about” means encompassing plus or minus 10%. For example, about 200 nucleotides refers to a range encompassing between 180 and 220 nucleotides.

As used herein, the term “amplicon” or “bioagent identifying amplicon” refers to a nucleic acid generated using the primer pairs described herein. The amplicon is typically double stranded DNA; however, it may be RNA and/or DNA:RNA. In some embodiments, the amplicon comprises DNA complementary to tick-borne pathogen and/or or endosymbiont RNA. In some embodiments, the amplicon comprises the sequences of the conserved regions/primer pairs and the intervening variable region. As discussed herein, primer pairs are configured to generate amplicons from two or more bioagents. As such, the base composition of any given amplicon may include the primer pair, the complement of the primer pair, the conserved regions and the variable region from the bioagent that was amplified to generate the amplicon. One skilled in the art understands that the incorporation of the designed primer pair sequences into an amplicon may replace the native bioagent sequences at the primer binding site, and complement thereof. After amplification of the target region using the primers the resultant amplicons having the primer sequences are used to generate the molecular mass data. Such is accounted for when identifying one or more bioagents using any particular primer pair. The amplicon further comprises a length that is compatible with mass spectrometry analysis. Bioagent identifying amplicons generate base compositions that are preferably unique to the identity of a bioagent.

Amplicons typically comprise from about 45 to about 200 consecutive nucleobases (i.e., from about 45 to about 200 μlinked nucleosides). One of ordinary skill in the art will appreciate that this range expressly embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One ordinarily skilled in the art will further appreciate that the above range is not an absolute limit to the length of an amplicon, but instead represents a preferred length range. Amplicons lengths falling outside of this range are also included herein so long as the amplicon is amenable to calculation of a base composition signature as herein described.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “base composition” refers to the number of each residue comprised in an amplicon or other nucleic acid, without consideration for the linear arrangement of these residues in the strand(s) of the amplicon. The amplicon residues comprise, adenosine (A), guanosine (G), cytidine, (C), (deoxy)thymidine (T), uracil (U), inosine (I), nitroindoles such as 5-nitroindole or 3-nitropyrrole, dP or dK (Hill et al.), an acyclic nucleoside analog containing 5-nitroindazole (Van Aerschot et al., Nucleosides and Nucleotides, 1995, 14, 1053-1056), the purine analog 1-(2-deoxy-β-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C. In some embodiments, the non-natural nucleosides used herein include 5-propynyluracil, 5-propynylcytosine and inosine. Herein the base composition for an unmodified DNA amplicon is notated as A_{w}GₓC_{y}T_{z}, wherein w, x, y and z are each independently a whole number representing the number of said nucleoside residues in an amplicon. Base compositions for amplicons comprising modified nucleosides are similarly notated to indicate the number of said natural and modified nucleosides in an amplicon. Base compositions are calculated from a molecular mass measurement of an amplicon, as described below. The calculated base composition for any given amplicon is then compared to a database of base compositions. A match between the calculated base composition and a single database entry reveals the identity of the bioagent.

As used herein, a “base composition probability cloud” is a representation of the diversity in base composition resulting from a variation in sequence that occurs among different isolates of a given species, family or genus. Base composition calculations for a plurality of amplicons are mapped on a pseudo four-dimensional plot. Related members in a family, genus or species typically cluster within this plot, forming a base composition probability cloud.

As used herein, the term “base composition signature” refers to the base composition generated by any one particular amplicon.

As used herein, a “bioagent” means any microorganism or infectious substance, or any naturally occurring, bioengineered or synthesized component of any such microorganism or infectious substance or any nucleic acid derived from any such microorganism or infectious substance. Those of ordinary skill in the art will understand fully what is meant by the term bioagent given the instant disclosure. Still, a non-exhaustive list of bioagents includes: cells, cell lines, human clinical samples, mammalian blood samples, cell cultures, bacterial cells, viruses, viroids, fungi, protists, parasites, rickettsiae, protozoa, animals, mammals or humans. Samples may be alive, non-replicating or dead or in a vegetative state (for example, vegetative bacteria or spores). In some embodiments, the bioagent is a tick-borne pathogen and/or or endosymbiont (e.g., including, but not limited to, a Spirochete, an alphaproteobacteria, a Francisella sp., Coxiella burnetii, a Flavivirus, Borrelia miyamotoi, Borrelia burgdorferi type 1, Borrelia burgdorferi type 2, Borrelia lonestari, Spotted Fever Group Rickettsia variant 1, Spotted Fever Group Rickettsia variant 2, Spotted Fever Group Rickettsia variant 3, Rickettsia amblyommii, Anaplasma phagocytophilum, Spiroplasma ixodetis, Ehrlichia variant 1, Wolbachia variant 1, Bartonella variant 1, Bartonella variant 2, Bartonella variant 3, Francisella genus or combinations thereof.

As used herein, a “bioagent division” is defined as group of bioagents above the species level and includes but is not limited to, orders, families, genus, classes, clades, genera or other such groupings of bioagents above the species level.

As used herein, “broad range survey primers” are intelligent primers designed to identify an unknown bioagent as a member of a particular biological division (e.g., an order, family, class, clade, or genus). However, in some cases the broad range survey primers are also able to identify unknown bioagents at the species or sub-species level. As used herein, “division-wide primers” are intelligent primers designed to identify a bioagent at the species level and “drill-down” primers are intelligent primers designed to identify a bioagent at the sub-species level. As used herein, the “sub-species” level of identification includes, but is not limited to, strains, subtypes, variants, and isolates. Drill-down primers are not always required for identification at the sub-species level because broad range survey intelligent primers may, in some cases provide sufficient identification resolution to accomplishing this identification objective.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “conserved region” in the context of nucleic acids refers to a nucleobase sequence (e.g., a subsequence of a nucleic acid, etc.) that is the same or similar in two or more different regions or segments of a given nucleic acid molecule (e.g., an intramolecular conserved region), or that is the same or similar in two or more different nucleic acid molecules (e.g., an intermolecular conserved region). To illustrate, a conserved region may be present in two or more different taxonomic ranks (e.g., two or more different genera, two or more different species, two or more different subspecies, and the like) or in two or more different nucleic acid molecules from the same organism. To further illustrate, in certain embodiments, nucleic acids comprising at least one conserved region typically have between about 70%-100%, between about 80-100%, between about 90-100%, between about 95-100%, or between about 99-100% sequence identity in that conserved region.

The term “correlates” refers to establishing a relationship between two or more things. In certain embodiments, for example, detected molecular masses of one or more amplicons indicate the presence or identity of a given bioagent in a sample. In some embodiments, base compositions are calculated or otherwise determined from the detected molecular masses of amplicons, which base compositions indicate the presence or identity of a given bioagent in a sample.

As used herein, in some embodiments the term “database” is used to refer to a collection of base composition molecular mass data. In other embodiments the term “database” is used to refer to a collection of base composition data. The base composition data in the database is indexed to bioagents and to primer pairs. The base composition data reported in the database comprises the number of each nucleoside in an amplicon that would be generated for each bioagent using each primer. The database can be populated by empirical data. In this aspect of populating the database, a bioagent is selected and a primer pair is used to generate an amplicon. The amplicon's molecular mass is determined using a mass spectrometer and the base composition calculated therefrom without sequencing i.e., without determining the linear sequence of nucleobases comprising the amplicon. Note that base composition entries in the database may be derived from sequencing data (e.g., known sequence data), but the base composition of the amplicon to be identified is determined without sequencing the amplicon. An entry in the database is made to associate correlate the base composition with the bioagent and the primer pair used. The database may also be populated using other databases comprising bioagent information. For example, using the GenBank database it is possible to perform electronic PCR using an electronic representation of a primer pair. This in silico method may provide the base composition for any or all selected bioagent(s) stored in the GenBank database. The information may then be used to populate the base composition database as described above. A base composition database can be in silico, a written table, a reference book, a spreadsheet or any form generally amenable to databases. Preferably, it is in silico on computer readable media.

The term “detect”, “detecting” or “detection” refers to an act of determining the existence or presence of one or more targets (e.g., bioagent nucleic acids, amplicons, etc.) in a sample.

As used herein, the term “etiology” refers to the causes or origins, of diseases or abnormal physiological conditions.

As used herein, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to nucleic acid sequences that are not found naturally associated with the gene sequences in the chromosome or are associated with portions of the chromosome not found in nature (e.g., genes expressed in loci where the gene is not normally expressed).

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. In context of the present invention, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers of the present invention, sequence identity is properly determined when the alignment is designated as Plus/Plus. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, “housekeeping gene” or “core viral gene” refers to a gene encoding a protein or RNA involved in basic functions required for survival and reproduction of a bioagent. Housekeeping genes include, but are not limited to, genes encoding RNA or proteins involved in translation, replication, recombination and repair, transcription, nucleotide metabolism, amino acid metabolism, lipid metabolism, energy generation, uptake, secretion and the like.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, “intelligent primers” or “primers” or “primer pairs” are oligonucleotides that are designed to bind to conserved sequence regions of two or more bioagent nucleic acid to generate bioagent identifying amplicons. In some embodiments, the bound primers flank an intervening variable region between the conserved binding sequences. Upon amplification, the primer pairs yield amplicons i.e., amplification products that provide base composition variability between the two or more bioagents. The variability of the base compositions allows for the identification of one or more individual bioagents from, e.g., two or more bioagents based on the base composition distinctions. The primer pairs are also configured to generate amplicons amenable to molecular mass analysis. Primer pair nomenclature, as used herein, includes naming a reference sequence. For example, the forward primer for primer pair number 3758 is named GENOME5UTR_NC001472-1-7389_{—}445_{—}463_F. The reference sequence that this primer is referring to is GenBank Accession No: NC_{—}001472 (first entered Aug. 1, 2000) (SEQ ID NO: 1). This primer is the forward primer of the pair (as denoted by “_F”) and it hybridizes with residues 445-463 of the reference sequence (445_{—}463), of the referenced Human Enterovirus B. The primer pairs are selected and configured in some embodiments, however, to hybridize with two or more bioagents. So, the nomenclature used is merely to provide a reference sequence, and not to indicate that the primers hybridize with and generate a bioagent identifying amplicon only from the reference sequence. Further, the sequences of the primer members of the primer pairs are not necessarily fully complementary to the conserved region of the reference bioagent. Rather, the sequences are designed to be “best fit” amongst a plurality of bioagents at these conserved binding sequences. Therefore, the primer members of the primer pairs have substantial complementarity with the conserved regions of the bioagents, including the reference bioagent.

As used herein, the term “molecular mass” refers to the mass of a compound as determined using mass spectrometry, specifically ESI-MS. Herein, the compound is preferably a nucleic acid, more preferably a double stranded nucleic acid, still more preferably a double stranded DNA nucleic acid and is most preferably an amplicon. When the nucleic acid is double stranded the molecular mass is determined for both strands. In one embodiment, the strands may be separated before introduction into the mass spectrometer, or the strands may be separated by the mass spectrometer (for example, electro-spray ionization will separate the hybridized strands). The molecular mass of each strand is measured by the mass spectrometer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N-6-methyladenosine, aziridinylcytosine, pseudoisocytosine, 5 (carboxyhydroxyl-methyl) uracil, 5-fluorouracil, 5 bromouracil, 5-carboxymethylaminomethyl 2 thiouracil, 5 carboxymethyl-aminomethyluracil, dihydrouracil, inosine, N6 isopentenyladenine, 1 methyladenine, 1-methylpseudo-uracil, 1 methylguanine, 1 methylinosine, 2,2-dimethyl-guanine, 2 methyladenine, 2 methylguanine, 3-methyl-cytosine, 5 methylcytosine, N6 methyladenine, 7 methylguanine, 5 methylaminomethyluracil, 5-methoxy-amino-methyl 2 thiouracil, beta D mannosylqueosine, 5′ methoxycarbonylmethyluracil, 5 methoxyuracil, 2 methylthio N6 isopentenyladenine, uracil 5 oxyacetic acid methylester, uracil 5 oxyacetic acid, oxybutoxosine, pseudouracil, queosine, 2 thiocytosine, 5-methyl-2 thiouracil, 2-thiouracil, 4 thiouracil, 5-methyluracil, N-uracil 5 oxyacetic acid methylester, uracil 5 oxyacetic acid, pseudouracil, queosine, 2-thiocytosine, and 2,6 diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetrahedron Lett. 22:1859-1862; the triester method of Matteucci et al. (1981) J. Am. Chem. Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

The term “probe nucleic acid” or “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using a probe under sufficiently stringent hybridization conditions permits the selective detection of a specific target sequence. The term “hybridizing region” refers to that region of a nucleic acid that is exactly or substantially complementary to, and therefore capable of hybridizing to, the target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support. A probe is generally included in a nucleic acid that comprises one or more labels (e.g., donor moieties, acceptor moieties, and/or quencher moieties), such as a 5′-nuclease probe, a hybridization probe, a fluorescent resonance energy transfer (FRET) probe, a hairpin probe, or a molecular beacon, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially or substantially complementary to one another); stable hybridization complexes may contain mismatched bases or unmatched bases. Modification of the stringent conditions may be necessary to permit a stable hybridization complex with one or more base pair mismatches or unmatched bases. Sambrook et al., Molecular Cloning: A Laboratory Manual, 3rd Ed., Cold Spring Harbor Laboratory Press, Cold Spring Harbor, N.Y. (2001), which is incorporated by reference, provides guidance for suitable modification. Stability of the target/probe hybridization complex depends on a number of variables including length of the oligonucleotide, base composition and sequence of the oligonucleotide, temperature, and ionic conditions. One of skill in the art will recognize that, in general, the exact complement of a given probe is similarly useful as a probe. One of skill in the art will also recognize that, in certain embodiments, probe nucleic acids can also be used as primer nucleic acids.

In some embodiments of the invention, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein a “sample” refers to anything capable of being analyzed by the methods provided herein. In some embodiments, the sample comprises or is suspected one or more nucleic acids capable of analysis by the methods. Preferably, the samples comprise nucleic acids (e.g., RNA, cDNAs, etc.) from one or more tick-borne pathogens and/or or endosymbionts. Samples can include, for example, evidence from a crime scene, blood, blood stains, semen, semen stains, bone, teeth, hair saliva, urine, feces, fingernails, muscle tissue, cigarettes, stamps, envelopes, dandruff, fingerprints, personal items, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As is used herein, the term “single primer pair identification” means that one or more bioagents can be identified using a single primer pair. A base composition signature for an amplicon may singly identify one or more bioagents.

As used herein, a “sub-species characteristic” is a genetic characteristic that provides the means to distinguish two members of the same bioagent species. For example, one bacterial or viral strain may be distinguished from another strain of the same species by possessing a genetic change (e.g., for example, a nucleotide deletion, addition or substitution) in one of the bacterial or viral genes.

As used herein, in some embodiments the term “substantial complementarity” means that a primer member of a primer pair comprises between about 70%-100%, or between about 80-100%, or between about 90-100%, or between about 95-100%, or between about 99-100% complementarity with the conserved binding sequence of a nucleic acid from a given bioagent. Similarly, the primer pairs provided herein may comprise between about 70%-100%, or between about 80-100%, or between about 90-100%, or between about 95-100% identity, or between about 99-100% sequence identity with the primer pairs disclosed in Tables 2, 7, 8, and 9. These ranges of complementarity and identity are inclusive of all whole or partial numbers embraced within the recited range numbers. For example, and not limitation, 75.667%, 82%, 91.2435% and 97% complementarity or sequence identity are all numbers that fall within the above recited range of 70% to 100%, therefore forming a part of this description. In some embodiments, any oligonucleotide primer pair may have one or both primers with less then 70% sequence homology with a corresponding member of any of the primer pairs of Tables 1 and 2 if the primer pair has the capability of producing an amplification product corresponding to the desired bioagent identifying amplicon.

A “system” in the context of analytical instrumentation refers a group of objects and/or devices that form a network for performing a desired objective.

As used herein, “triangulation identification” means the use of more than one primer pair to generate a corresponding amplicon for identification of a bioagent. The more than one primer pair can be used in individual wells or vessels or in a multiplex PCR assay. Alternatively, PCR reactions may be carried out in single wells or vessels comprising a different primer pair in each well or vessel. Following amplification the amplicons are pooled into a single well or container which is then subjected to molecular mass analysis. The combination of pooled amplicons can be chosen such that the expected ranges of molecular masses of individual amplicons are not overlapping and thus will not complicate identification of signals. Triangulation is a process of elimination, wherein a first primer pair identifies that an unknown bioagent may be one of a group of bioagents. Subsequent primer pairs are used in triangulation identification to further refine the identity of the bioagent amongst the subset of possibilities generated with the earlier primer pair. Triangulation identification is complete when the identity of the bioagent is determined. The triangulation identification process may also be used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected compositions from the B. anthracis genome would suggest a genetic engineering event.

As used herein, the term “unknown bioagent” can mean, for example: (i) a bioagent whose existence is not known (for example, the SARS coronavirus was unknown prior to April 2003) and/or (ii) a bioagent whose existence is known (such as the well known bacterial species Staphylococcus aureus for example) but which is not known to be in a sample to be analyzed. For example, if the method for identification of coronaviruses disclosed in commonly owned U.S. patent Ser. No. 10/829,826 (incorporated herein by reference in its entirety) was to be employed prior to April 2003 to identify the SARS coronavirus in a clinical sample, both meanings of “unknown” bioagent are applicable since the SARS coronavirus was unknown to science prior to April, 2003 and since it was not known what bioagent (in this case a coronavirus) was present in the sample. On the other hand, if the method of U.S. patent Ser. No. 10/829,826 was to be employed subsequent to April 2003 to identify the SARS coronavirus in a clinical sample, the second meaning (ii) of “unknown” bioagent would apply because the SARS coronavirus became known to science subsequent to April 2003 because it was not known what bioagent was present in the sample.

As used herein, the term “variable region” is used to describe a region that falls between any one primer pair described herein. The region possesses distinct base compositions between at least two bioagents, such that at least one bioagent can be identified at the family, genus, species or sub-species level. The degree of variability between the at least two bioagents need only be sufficient to allow for identification using mass spectrometry analysis, as described herein.

As used herein, “viral nucleic acid” includes, but is not limited to, DNA, RNA, or DNA that has been obtained from viral RNA, such as, for example, by performing a reverse transcription reaction. Viral RNA can either be single-stranded (of positive or negative polarity) or double-stranded.

As used herein, a “wobble base” is a variation in a codon found at the third nucleotide position of a DNA triplet. Variations in conserved regions of sequence are often found at the third nucleotide position due to redundancy in the amino acid code.

Provided herein are methods, compositions, kits, and related systems for the detection and identification of bioagents using bioagent identifying amplicons. In overview, primers may be selected to hybridize to conserved sequence regions of nucleic acids derived from a bioagent and which bracket variable sequence regions to yield a bioagent identifying amplicon which can be amplified and which is amenable to molecular mass determination. The molecular mass is typically converted to a base composition, which indicates the number of each nucleotide in the amplicon. The molecular mass or corresponding base composition signature of the amplicon is then typically queried against a database of molecular masses or base composition signatures indexed to bioagents and to the primer pair used to generate the amplicon. A match of the measured base composition to a database entry base composition associates the sample bioagent to an indexed bioagent in the database. Thus, the identity of the unknown bioagent is determined in certain embodiments. Prior knowledge of the unknown bioagent is not necessary. In some instances, the measured base composition associates with more than one database entry base composition. Thus, a second/subsequent primer pair is generally used to generate an amplicon, and its measured base composition is similarly compared to the database to determine its identity in triangulation identification. Furthermore, the methods and other aspects of the invention can be applied to rapid parallel multiplex analyses, the results of which can be employed in a triangulation identification strategy. The present invention provides rapid throughput and does not require nucleic acid sequencing of the amplified target sequence for bioagent detection and identification. Exemplary compositions and methods of embodiments of the present invention are described below.

Since genetic data provide the underlying basis for identification of bioagents, it is generally necessary to select segments or regions of nucleic acids which provide sufficient variability to distinguish individual bioagents and whose molecular mass is amenable to molecular mass determination.

In some embodiments, at least one tick-borne or endosymbiont nucleic acid segment is amplified in the process of identifying the bioagent. Thus, the nucleic acid segments that can be amplified by the primers disclosed herein and that provide sufficient variability to distinguish individual bioagents and whose molecular masses are amenable to molecular mass determination are herein described as bioagent identifying amplicons. In certain embodiments, tick-borne or endosymbiont bioagents are identified via amplicons generated with the primers described herein using methods of detection other than molecular mass-based detection, such as real-time PCR (e.g., using 5′-nuclease probes, hairpin probes, hybridization probes, nucleic acid binding dyes, or the like) or other approaches known to persons of skill in the art.

In some embodiments, it is the combination of the portions of the bioagent nucleic acid segment to which the primers hybridize (hybridization sites) and the variable region between the primer hybridization sites that comprises the bioagent identifying amplicon.

In certain embodiments, bioagent identifying amplicons amenable to molecular mass determination which are produced by the primers described herein are either of a length, size or mass compatible with the particular mode of molecular mass determination or compatible with a means of providing a predictable fragmentation pattern in order to obtain predictable fragments of a length compatible with the particular mode of molecular mass determination. Such means of providing a predictable fragmentation pattern of an amplicon include, but are not limited to, cleavage with restriction enzymes or cleavage primers, sonication or other means of fragmentation. Thus, in some embodiments, bioagent identifying amplicons are larger than 200 nucleobases and are amenable to molecular mass determination following restriction digestion. Methods of using restriction enzymes and cleavage primers are well known to those with ordinary skill in the art.

In some embodiments, amplicons corresponding to bioagent identifying amplicons are obtained using the polymerase chain reaction (PCR) which is a routine method to those with ordinary skill in the molecular biology arts. Other amplification methods may be used such as ligase chain reaction (LCR), low-stringency single primer PCR, and multiple strand displacement amplification (MDA). These methods are also known to those with ordinary skill. (Michael, SF., Biotechniques (1994), 16:411-412 and Dean et al., Proc. Natl. Acad. Sci. U.S.A. (2002), 99, 5261-5266).

In some embodiments, for each group of organisms, candidate target sequences are identified from which nucleotide alignments are created and analyzed. Primers are then configured by selecting priming regions to facilitate the selection of candidate primer pairs. The primer pair sequence is typically a “best fit” amongst the aligned sequences, such that the primer pair sequence may or may not be fully complementary to the hybridization region on any one of the bioagents in the alignment. Thus, best fit primer pair sequences are those with sufficient complementarity with two or more bioagents to hybridize with the two or more bioagents and generate an amplicon. The primer pairs are then subjected to in silico analysis by electronic PCR (ePCR) wherein bioagent identifying amplicons are obtained from sequence databases such as GenBank or other sequence collections and tested for specificity in silico. Bioagent identifying amplicons obtained from ePCR of GenBank sequences may also be analyzed by a probability model which predicts the capability of a given amplicon to identify unknown bioagents. Preferably, the base compositions of amplicons with favorable probability scores are then stored in a base composition database. Alternatively, base compositions of the bioagent identifying amplicons obtained from the primers and GenBank sequences are directly entered into the base composition database. Candidate primer pairs are validated by in vitro amplification by a method such as PCR analysis of nucleic acid from a collection of organisms. Amplicons thus obtained are analyzed to confirm the sensitivity, specificity and reproducibility of the primers used to obtain the amplicons.

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The primers typically are employed as compositions for use in methods for identification of bioagents as follows: a primer pair composition is contacted with nucleic acid (such as, for example, DNA, or DNA reverse transcribed from the RNA of an RNA virus) of an unknown bioagent. The nucleic acid is then amplified by a nucleic acid amplification technique, such as PCR for example, to obtain an amplicon that represents a bioagent identifying amplicon. The molecular mass of the strands of the double-stranded amplicon is determined by a molecular mass measurement technique such as mass spectrometry, for example. Preferably the two strands of the double-stranded amplicon are separated during the ionization process; however, they may be separated prior to mass spectrometry measurement. In some embodiments, the mass spectrometer is electrospray Fourier transform ion cyclotron resonance mass spectrometry (ESI-FTICR-MS) or electrospray time of flight mass spectrometry (ESI-TOF-MS). A list of possible base compositions may be generated for the molecular mass value obtained for each strand and the choice of the base composition from the list is facilitated by matching the base composition of one strand with a complementary base composition of the other strand. The measured molecular mass or base composition calculated therefrom is then compared with a database of molecular masses or base compositions indexed to primer pairs and to known bioagents. A match between the measured molecular mass or base composition of the amplicon and the database molecular mass or base composition for that indexed primer pair will correlate the measured molecular mass or base composition with an indexed bioagent, thus identifying the unknown bioagent. In some embodiments, the primer pair used is at least one of the primer pairs of Tables 1 or 2. In some embodiments, the method is repeated using a different primer pair to resolve possible ambiguities in the identification process or to improve the confidence level for the identification assignment (triangulation identification).

In some embodiments, a bioagent identifying amplicon may be produced using only a single primer (either the forward or reverse primer of any given primer pair), provided an appropriate amplification method is chosen, such as, for example, low stringency single primer PCR (LSSP-PCR).

In some embodiments, the oligonucleotide primers are broad range survey primers which hybridize to conserved regions of nucleic acid encoding a gene that is common to a class of bioagents (e.g., tick-borne or endosymbiont), though the sequences vary. The broad range primer may identify the unknown bioagent, depending on which bioagent is in the sample. In other cases, the molecular mass or base composition of an amplicon does not provide sufficient resolution to identify the unknown bioagent as any one bioagent at or below the species level. These cases generally benefit from further analysis of one or more amplicons generated from at least one additional broad range survey primer pair or from at least one additional division-wide primer pair, or from at least one additional drill-down primer pair. Identification of sub-species characteristics may be needed for determining proper clinical treatment of tick-borne infections, or in rapidly responding to an outbreak of a new tick-borne pathogen strain to prevent massive epidemic or pandemic.

In some embodiments, the primers used for amplification hybridize to and amplify genomic DNA, DNA of bacterial plasmids, DNA of DNA viruses or DNA reverse transcribed from RNA of an RNA virus.

In some embodiments, the primers used for amplification hybridize directly to RNA (e.g., viral RNA) and act as reverse transcription primers for obtaining DNA from direct amplification of RNA. Methods of amplifying RNA to produce cDNA using reverse transcriptase are well known to those with ordinary skill in the art and can be routinely established without undue experimentation.

One with ordinary skill in the art of design of amplification primers will recognize that a given primer need not hybridize with 100% complementarity in order to effectively prime the synthesis of a complementary nucleic acid strand in an amplification reaction. Primer pair sequences may be a “best fit” amongst the aligned bioagent sequences, thus not be fully complementary to the hybridization region on any one of the bioagents in the alignment. Moreover, a primer may hybridize over one or more segments such that intervening or adjacent segments are not involved in the hybridization event (e.g., for example, a loop structure or a hairpin structure). The primers may comprise at least 70%, at least 75%, at least 80%, at least 85%, at least 90%, at least 95% or at least 99% sequence identity with any of the primers listed in Tables 1 and 2. Thus, in some embodiments, an extent of variation of 70% to 100%, or any range falling within, of the sequence identity is possible relative to the specific primer sequences disclosed herein. To illustrate, determination of sequence identity is described in the following example: a primer 20 nucleobases in length which is identical to another 20 nucleobase primer having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer. Percent identity need not be a whole number, for example when a 28 consecutive nucleobase primer is completely identical to a 31 consecutive nucleobase primer (28/31=0.9032 or 90.3% identical).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions of nucleic acid, is between about 70% and about 80%. In other embodiments, homology, sequence identity or complementarity, is between about 80% and about 90%. In yet other embodiments, homology, sequence identity or complementarity, is at least 90%, at least 92%, at least 94%, at least 95%, at least 96%, at least 97%, at least 98%, at least 99% or is 100%.

In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range falling within) sequence identity with the primer sequences specifically disclosed herein.

One with ordinary skill is able to calculate percent sequence identity or percent sequence homology and is able to determine, without undue experimentation, the effects of variation of primer sequence identity on the function of the primer in its role in priming synthesis of a complementary strand of nucleic acid for production of an amplicon of a corresponding bioagent identifying amplicon.

In some embodiments, the oligonucleotide primers are 13 to 35 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin.

In some embodiments, any given primer comprises a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated A residues as a result of the non-specific enzyme activity of, e.g., Taq DNA polymerase (Magnuson et al., Biotechniques, 1996, 21, 700-709), an occurrence which may lead to ambiguous results arising from molecular mass analysis.

Primers may contain one or more universal bases. Because any variation (due to codon wobble in the third position) in the conserved regions among species is likely to occur in the third position of a DNA (or RNA) triplet, oligonucleotide primers can be designed such that the nucleotide corresponding to this position is a base which can bind to more than one nucleotide, referred to herein as a “universal nucleobase.” For example, under this “wobble” pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides, 1995, 14, 1001-1003), the degenerate nucleotides dP or dK (Hill et al.), an acyclic nucleoside analog containing 5-nitroindazole (Van Aerschot et al., Nucleosides and Nucleotides, 1995, 14, 1053-1056) or the purine analog 1-(2-deoxy-.beta.-D-ribofuranosyl)-imidazole-4-carboxamide (Sala et al., Nucl. Acids Res., 1996, 24, 3302-3306).

In some embodiments, to compensate for weaker binding by the wobble base, the oligonucleotide primers are configured such that the first and second positions of each triplet are occupied by nucleotide analogs which bind with greater affinity than the unmodified nucleotide. Examples of these analogs include, but are not limited to, 2,6-diaminopurine which binds to thymine, 5-propynyluracil which binds to adenine and 5-propynylcytosine and phenoxazines, including G-clamp, which binds to G. Propynylated pyrimidines are described in U.S. Pat. Nos. 5,645,985, 5,830,653 and 5,484,908, each of which is commonly owned and incorporated herein by reference in its entirety. Propynylated primers are described in U.S Pre-Grant Publication No. 2003-0170682; also commonly owned and incorporated herein by reference in its entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety.

In some embodiments, primer hybridization is enhanced using primers and probes containing 5-propynyl deoxy-cytidine and deoxy-thymidine nucleotides. These modified primers offer increased affinity and base pairing selectivity.

In some embodiments, non-template primer tags are used to increase the melting temperature (Tₘ) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers contain mass-modifying tags. Reducing the total number of possible base compositions of a nucleic acid of specific molecular weight provides a means of avoiding a possible source of ambiguity in determination of base composition of amplicons. Addition of mass-modifying tags to certain nucleobases of a given primer will result in simplification of de novo determination of base composition of a given bioagent identifying amplicon from its molecular mass.

In some embodiments, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹³N and ¹³C.

In some embodiments, the molecular mass of a given bioagent identifying amplicon is determined by mass spectrometry. Mass spectrometry is intrinsically a parallel detection scheme without the need for radioactive or fluorescent labels, since every amplicon is identified by its molecular mass. The current state of the art in mass spectrometry is such that less than femtomole quantities of material can be readily analyzed to afford information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplicons using one of a variety of ionization techniques to convert the sample to the gas phase. These ionization methods include, but are not limited to, electrospray ionization (ESI), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, assignment of previously unobserved base compositions (also known as “true unknown base compositions”) to a given phylogeny can be accomplished via the use of pattern classifier model algorithms. Base compositions, like sequences, may vary slightly from strain to strain within species, for example. In some embodiments, the pattern classifier model is the mutational probability model. In other embodiments, the pattern classifier is the polytope model. A polytope model is the mutational probability model that incorporates both the restrictions among strains and position dependence of a given nucleobase within a triplet. In certain embodiments, a polytope pattern classifier is used to classify a test or unknown organism according to its amplicon base composition.

In some embodiments, it is possible to manage this diversity by building “base composition probability clouds” around the composition constraints for each species. A “pseudo four-dimensional plot” may be used to visualize the concept of base composition probability clouds. Optimal primer design typically involves an optimal choice of bioagent identifying amplicons and maximizes the separation between the base composition signatures of individual bioagents. Areas where clouds overlap generally indicate regions that may result in a misclassification, a problem which is overcome by a triangulation identification process using bioagent identifying amplicons not affected by overlap of base composition probability clouds.

In some embodiments, base composition probability clouds provide the means for screening potential primer pairs in order to avoid potential misclassifications of base compositions. In other embodiments, base composition probability clouds provide the means for predicting the identity of an unknown bioagent whose assigned base composition was not previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

Provided herein is bioagent classifying information at a level sufficient to identify a given bioagent. Furthermore, the process of determining a previously unknown base composition for a given bioagent (for example, in a case where sequence information is unavailable) has utility by providing additional bioagent indexing information with which to populate base composition databases. The process of future bioagent identification is thus improved as additional base composition signature indexes become available in base composition databases.

In some embodiments, the identity and quantity of an unknown bioagent may be determined by adding primers and a known quantity of a calibration polynucleotide to a sample containing nucleic acid of an unknown bioagent. The total nucleic acid in the sample is then subjected to an amplification reaction to obtain amplicons. The molecular masses of amplicons are determined from which are obtained molecular mass and abundance data. The molecular mass of the bioagent identifying amplicon provides for its identification and the molecular mass of the calibration amplicon obtained from the calibration polynucleotide provides for its quantification. The abundance data of the bioagent identifying amplicon is recorded and the abundance data for the calibration data is recorded, both of which are used in a calculation which determines the quantity of unknown bioagent in the sample.

In certain embodiments, a sample comprising an unknown bioagent is contacted with a primer pair which amplifies the nucleic acid from the bioagent, and a known quantity of a polynucleotide that comprises a calibration sequence. The rate of amplification is reasonably assumed to be similar for the nucleic acid of the bioagent and for the calibration sequence. The amplification reaction then produces two amplicons: a bioagent identifying amplicon and a calibration amplicon. The bioagent identifying amplicon and the calibration amplicon are distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2-8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent by base composition analysis. The abundance data enables calculation of the quantity of the bioagent, based on the knowledge of the quantity of calibration polynucleotide contacted with the sample.

In some embodiments, construction of a standard curve in which the amount of calibration or calibrant polynucleotide spiked into the sample is varied provides additional resolution and improved confidence for the determination of the quantity of bioagent in the sample. The use of standard curves for analytical determination of molecular quantities is well known to one with ordinary skill and can be performed without undue experimentation. Alternatively, the calibration polynucleotide can be amplified in its own PCR reaction vessel or vessels under the same conditions as the bioagent. A standard curve may be prepared there from, and the relative abundance of the bioagent determined by methods such as linear regression. In some embodiments, multiplex amplification is performed where multiple bioagent identifying amplicons are amplified with multiple primer pairs which also amplify the corresponding standard calibration sequences. In this or other embodiments, the standard calibration sequences are optionally included within a single construct (preferably a vector) which functions as the calibration polynucleotide. Competitive PCR, quantitative PCR, quantitative competitive PCR, multiplex and calibration polynucleotides are all methods and materials well known to those ordinarily skilled in the art and can be performed without undue experimentation.

In some embodiments, the calibrant polynucleotide is used as an internal positive control to confirm that amplification conditions and subsequent analysis steps are successful in producing a measurable amplicon. Even in the absence of copies of the genome of a bioagent, the calibration polynucleotide should give rise to a calibration amplicon. Failure to produce a measurable calibration amplicon indicates a failure of amplification or subsequent analysis step such as amplicon purification or molecular mass determination. Reaching a conclusion that such failures have occurred is, in itself, a useful event. In some embodiments, the calibration sequence is comprised of DNA. In some embodiments, the calibration sequence is comprised of RNA.

In some embodiments, a calibration sequence is inserted into a vector which then functions as the calibration polynucleotide. In some embodiments, more than one calibration sequence is inserted into the vector that functions as the calibration polynucleotide. Such a calibration polynucleotide is herein termed a “combination calibration polynucleotide.” The process of inserting polynucleotides into vectors is routine to those skilled in the art, and may be accomplished without undue experimentation. Thus, it should be recognized that the calibration method should not be limited to the embodiments described herein. The calibration method can be applied for determination of the quantity of any bioagent identifying amplicon when an appropriate standard calibrant polynucleotide sequence is designed and used. The process of choosing an appropriate vector for insertion of a calibrant is also a routine operation that can be accomplished by one with ordinary skill without undue experimentation.

In certain embodiments, primer pairs are configured to produce bioagent identifying amplicons within more conserved regions of tick-borne or endosymbiont bioagents while others produce bioagent identifying amplicons within regions that are may evolve more quickly. Primer pairs that characterize amplicons in a conserved region with low probability that the region will evolve past the point of primer recognition are useful, e.g., as a broad range survey-type primer. Primer pairs that characterize an amplicon corresponding to an evolving genomic region are useful, e.g., for distinguishing emerging strain variants.

The primer pairs described herein provide reagents, e.g., for identifying diseases caused by emerging bioagents. Base composition analysis eliminates the need for prior knowledge of bioagent sequence to generate hybridization probes. Thus, in another embodiment, there is provided a method for determining the etiology of a tick-borne infection when the process of identification of bioagents is carried out in a clinical setting, and even when the bioagent is a new species. This is possible because the methods may not be confounded by naturally occurring evolutionary variations (a major concern when using probe based or sequencing dependent methods for characterizing bioagents that evolve rapidly). Measurement of molecular mass and determination of base composition is accomplished in an unbiased manner without sequence prejudice, and without the need for specificity as is required with probes.

Another embodiment provides a means of tracking the spread of any species or strain of bioagent when a plurality of samples obtained from different geographical locations are analyzed by methods described above in an epidemiological setting. For example, a plurality of samples from a plurality of different locations may be analyzed with primers which produce bioagent identifying amplicons, a subset of which contains a specific bioagent. The corresponding locations of the members of the bioagent-containing subset indicate the spread of the specific bioagent to the corresponding locations.

Also provided are kits for carrying out the methods described herein. In some embodiments, the kit may comprise a sufficient quantity of one or more primer pairs to perform an amplification reaction on a target polynucleotide from a bioagent to form a bioagent identifying amplicon. In some embodiments, the kit may comprise from one to fifty primer pairs, from one to twenty primer pairs, from one to ten primer pairs, from one to eight primer pairs or from two to five primer pairs. In some embodiments, the kit may comprise one or more primer pairs recited in Tables 1 and 2.

In some embodiments, the kit may comprise one or more broad range survey primer(s), division wide primer(s), or drill-down primer(s), or any combination thereof. A kit may be configured so as to comprise select primer pairs for identification of a particular bioagent. For example, a broad range survey primer kit may be used initially to identify an unknown bioagent as a member of a specific family of tick-borne or endosymbiont bioagent. Another example of a division-wide kit may be used to distinguish species or sub-types of tick-borne or endosymbiont bioagents. In some embodiments, the kit may contain standardized calibration polynucleotides for use as internal amplification calibrants.

In some embodiments, the kit may also comprise a sufficient quantity of reverse transcriptase (if an RNA virus is to be identified for example), a DNA polymerase, suitable nucleoside triphosphates (including any of those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. In some embodiments, the kit further comprises instructions for analysis, interpretation and dissemination of data acquired by the kit. In other embodiments, instructions for the operation, analysis, interpretation and dissemination of the data of the kit are provided on computer readable media. A kit may also comprise amplification reaction containers such as microcentrifuge tubes, microtiter plates, and the like. A kit may also comprise reagents or other materials for isolating bioagent nucleic acid or bioagent identifying amplicons from amplification, including, for example, detergents, solvents, or ion exchange resins which may be linked to magnetic beads. A kit may also comprise a table of measured or calculated molecular masses and/or base compositions of bioagents using the primer pairs of the kit.

The invention also provides systems that can be used to perform various assays relating to tick-borne or endosymbiont detection or identification. In certain embodiments, systems include mass spectrometers configured to detect molecular masses of amplicons produced using purified oligonucleotide primer pairs described herein. Other detectors that are optionally adapted for use in the systems of the invention are described further below. In some embodiments, systems also include controllers operably connected to mass spectrometers and/or other system components. In some of these embodiments, controllers are configured to correlate the molecular masses of the amplicons with tick-borne or endosymbiont bioagents to effect detection or identification (e.g., at genus, species, and/or sub-species levels). In some embodiments, controllers are configured to determine base compositions of the amplicons from the molecular masses of the amplicons. As described herein, the base compositions generally correspond to the tick-borne or endosymbiont bioagent identities. In certain embodiments, controllers include or are operably connected to databases of known molecular masses and/or known base compositions of amplicons of known tick-borne or endosymbiont bioagents produced with the primer pairs described herein. Controllers are described further below.

In some embodiments, systems include one or more of the primer pairs described herein (e.g., in Tables 1 and 2). In certain embodiments, the oligonucleotides are arrayed on solid supports, whereas in others, they are provided in one or more containers, e.g., for assays performed in solution. In certain embodiments, the systems also include at least one detector or detection component (e.g., a spectrometer) that is configured to detect detectable signals produced in the container or on the support. In addition, the systems also optionally include at least one thermal modulator (e.g., a thermal cycling device) operably connected to the containers or solid supports to modulate temperature in the containers or on the solid supports, and/or at least one fluid transfer component (e.g., an automated pipettor) that transfers fluid to and/or from the containers or solid supports, e.g., for performing one or more assays (e.g., nucleic acid amplification, real-time amplicon detection, etc.) in the containers or on the solid supports.

Detectors are typically structured to detect detectable signals produced, e.g., in or proximal to another component of the given assay system (e.g., in a container and/or on a solid support). Suitable signal detectors that are optionally utilized, or adapted for use, herein detect, e.g., fluorescence, phosphorescence, radioactivity, absorbance, refractive index, luminescence, or mass. Detectors optionally monitor one or a plurality of signals from upstream and/or downstream of the performance of, e.g., a given assay step. For example, detectors optionally monitor a plurality of optical signals, which correspond in position to “real-time” results. Example detectors or sensors include photomultiplier tubes, CCD arrays, optical sensors, temperature sensors, pressure sensors, pH sensors, conductivity sensors, or scanning detectors. Detectors are also described in, e.g., Skoog et al., Principles of Instrumental Analysis, 5^{th} Ed., Harcourt Brace College Publishers (1998), Curren, Analytical Instrumentation: Performance Characteristics and Quality, John Wiley & Sons, Inc. (2000), Sharma et al., Introduction to Fluorescence Spectroscopy, John Wiley & Sons, Inc. (1999), Valeur, Molecular Fluorescence: Principles and Applications, John Wiley & Sons, Inc. (2002), and Gore, Spectrophotometry and Spectrofluorimetry: A Practical Approach, 2.sup.nd Ed., Oxford University Press (2000), which are each incorporated by reference.

As mentioned above, the systems of the invention also typically include controllers that are operably connected to one or more components (e.g., detectors, databases, thermal modulators, fluid transfer components, robotic material handling devices, and the like) of the given system to control operation of the components. More specifically, controllers are generally included either as separate or integral system components that are utilized, e.g., to receive data from detectors (e.g., molecular masses, etc.), to effect and/or regulate temperature in the containers, to effect and/or regulate fluid flow to or from selected containers. Controllers and/or other system components are optionally coupled to an appropriately programmed processor, computer, digital device, information appliance, or other logic device (e.g., including an analog to digital or digital to analog converter as needed), which functions to instruct the operation of these instruments in accordance with preprogrammed or user input instructions, receive data and information from these instruments, and interpret, manipulate and report this information to the user. Suitable controllers are generally known in the art and are available from various commercial sources.

Any controller or computer optionally includes a monitor, which is often a cathode ray tube (“CRT”) display, a flat panel display (e.g., active matrix liquid crystal display or liquid crystal display), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. These components are illustrated further below.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation. The computer then receives the data from, e.g., sensors/detectors included within the system, and interprets the data, either provides it in a user understood format, or uses that data to initiate further controller instructions, in accordance with the programming.

As will be understood by practitioners in the art from the teachings provided herein, aspects of the invention are optionally implemented in hardware and/or software. In some embodiments, different aspects of the invention are implemented in either client-side logic or server-side logic. As will be understood in the art, the invention or components thereof may be embodied in a media program component (e.g., a fixed media component) containing logic instructions and/or data that, when loaded into an appropriately configured computing device, cause that device to perform as desired. As will also be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

While the present invention has been described with specificity in accordance with certain of its embodiments, the following examples serve only to illustrate the invention and are not intended to limit the same. In order that the invention disclosed herein may be more efficiently understood, examples are provided below. It should be understood that these examples are for illustrative purposes only and are not to be construed as limiting the invention in any manner.

### Example 1

### Tick-Borne Surveillance Assay

This Example describes a broad range detection assay to examine the microorganisms in ticks. Pathogen and endosymbionts diversity was examined. PCR/ESI-MS performed on the Ibis T5000 biosensor platform was used to detect microorganisms in ticks. A multi-locus genotyping assay with 18 PCR primer pairs targeting known pathogen families as well as broad range targets was developed. Embodiments of the invention can employ any one or more of the 18 primer pairs in any combination. Base composition signatures determined from these products were used to identify the organisms found in ticks.

### Methods and Results

### Tick-Borne Surveillance Assay

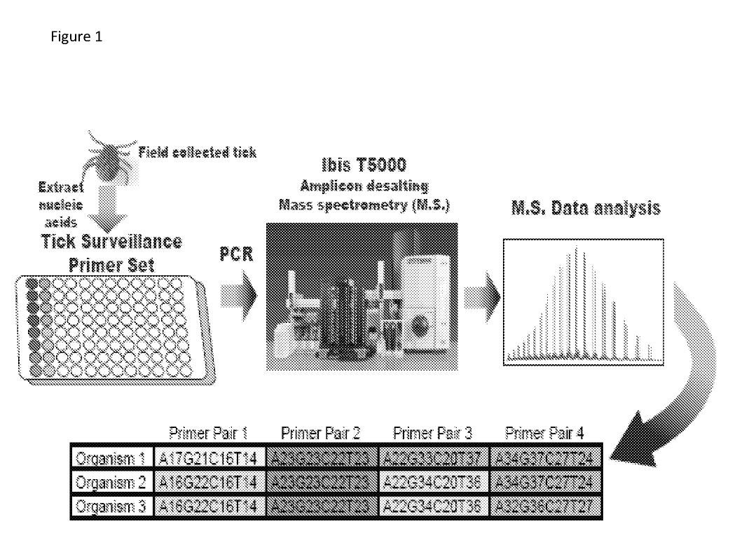

Tick nucleic acids are extracted as described below. PCR/ESI/MS was performed on the Ibis T5000 biosensor platform using the primer pairs described in Tables 1 and 2. The Ibis T5000 Biosensor system (Ibis Biosciences, Carlsbad, Calif.) comprises a barcode reader, a magnetic bead desalting module, an autosampler and a ESI-TOF mass spectrometer. The instrument is capable of a throughput of >1500 wells in 24 hours (Ecker et al., JALA 11:341 2006; Hofstadler et al., J. Mass Spectrom. 242:23 2005). A schematic of PCR/ESI-MS is shown in FIG. 1. Briefly, microbe variable genomic regions surrounded by conserved DNA are first identified for analysis. Next, a broad range primer is used to amplify the region of interest. Mass spectrometry is then used to analyze the composition of amplified nucleic acids. The organisms are then identified by using base composition fingerprints.

TABLE 1

| Primer Pair | Target Clade/Genus | Target |
|---|---|---|
| BCT3517 | All Spirochetes | flagellin |
| BCT3515 | Borrelia | rplB |
| BCT3511 | Borrelia | gyrB |
| BCT3519 | Borrelia | hbb |
| BCT1083 | Rickettsia | rnpA |
| BCT1084 | Rickettsia | rnpA |
| BCT3569 | Alphaproteobacteria | gltA |
| BCT3575 | Alphaproteobacteria | rpoB |
| BCT2328 | Francisella | asd |
| BCT2332 | Francisella | galE |
| BCT1079 | Coxiella burnetii | icd |
| BCT1080 | Coxiella burnetii | IS1111A |
| BCT3565 | Spiroplasma | rpoB |
| BCT3568 | Spiroplasma | S. kunkeii TraE-like gene |
| BCT348 | All bacteria | 16S rRNA gene |
| BCT360 | All bacteria | 23S rRNA gene |
| VIR2215 | All Flaviviruses | RdRp |
| VIR2217 | All Flaviviruses | RdRp |

TABLE 2

| | | primer | SEQ ID |
|---|---|---|---|
| primer code | primer sequence | pair code | NO |
| BCT8241F | TGCTGAAGAGCTTGGAATGCA | BCT3517 | 1 |
| BCT8245F | TCGAATAATGTTATTGAGTTTAGATCTTTTGGTAC | BCT3519 | 2 |
| BCT8229F | TGCATTTGAAAGCTTGGCATTGCC | BCT3511 | 3 |
| BCT8237F | TCCACAAGGTGGTGGTGAAGG | BCT3515 | 4 |
| BCT2764F | TAAGAGCGCACCGGTAAGTTGG | BCT1083 | 5 |
| BCT2765F | TCCACCAAGAGCAAGATCAAATAGGC | BCT1084 | 6 |
| BCT8334F | TGCATGCAGATCATGAACAAAATGC | BCT3569 | 7 |
| BCT8346F | TGCATCACTTGGTTGATGATAAGATACATGC | BCT3575 | 8 |
| BCT5602F | TGAGGGTTTTATGCTTAAAGTTGGTTTTATTGGTT | BCT2328 | 9 |
| BCT5610F | TCAGCTAGACCTTTTAGGTAAAGCTAAGCT | BCT2332 | 10 |
| BCT2717F | TCGCCGTGGAAAAATCCTACGCT | BCT1079 | 11 |
| BCT2719F | TCAGTATGTATCCACCGTAGCCAGTC | BCT1080 | 12 |
| BCT8326F | TGTATATGTTGAAACTATCTCACATGGTTGATGA | BCT3565 | 13 |
| BCT8332F | TGCTTTTATTGTTGGCCAGATGGG | BCT3568 | 14 |
| BCT1393F | TTTCGATGCAACGCGAAGAACCT | BCT348 | 15 |
| BCT1386F | TCTGTTCTTAGTACGAGAGGACC | BCT360 | 16 |
| VIR5394F | TAGCCGAGCCATCTGGTACATGTGG | VIR2215 | 17 |
| VIR5397F | TGTGTCTACAACATGATGGGAAAGAGAGA | VIR2217 | 18 |
| BCT8242R | TACAGCAATTGCTTCATCTTGATTTGC | BCT3517 | 19 |
| BCT8246R | TGGACGAAAATACGCAACATGATGATC | BCT3519 | 20 |
| BCT8230R | TCATTTTAGCACTTCCTCCAGCAGAATC | BCT3511 | 21 |
| BCT8238R | TCGGCTGTCCCCAAGGAG | BCT3515 | 22 |
| BCT2763R | TCAAGCGATCTACCCGCATTACAA | BCT1083 | 23 |
| BCT2763R | TCAAGCGATCTACCCGCATTACAA | BCT1084 | 24 |
| BCT8335R | TCCATGTGCTGGTCCCCA | BCT3569 | 25 |
| BCT8347R | TCACCAAAACGCTGACCACCAAA | BCT3575 | 26 |
| BCT5603R | TGATTCGATCATACGAGACATTAAAACTGAG | BCT2328 | 27 |
| BCT5625R | TCTCACCTACAGCTTTAAAGCCAGCAAAATG | BCT2332 | 28 |
| BCT2718R | TAGCCTTTTCTCCGGCGTAGATCT | BCT1079 | 29 |
| BCT2720R | TAAACGTCCGATACCAATGGTTCGCTC | BCT1080 | 30 |
| BCT8327R | TGTGCTTTTCCTCCAAATGGTTGTTG | BCT3565 | 31 |
| BCT8333R | TGGCAAAACTACCATATTCGCGTTG | BCT3568 | 32 |
| BCT1370R | TACGAGCTGACGACAGCCATG | BCT348 | 33 |
| BCT1402R | TTTCGTGCTTAGATGCTTTCAG | BCT360 | 34 |
| VIR5393R | TCTCTGGAAAGCCAGTGGTCTTCATT | VIR2215 | 35 |
| VIR5398R | TGCTCCCAGCCACATGTACCA | VIR2217 | 36 |

### Total Nucleic Acid Isolation

In order to detect and identify all the pathogens carried by ticks, RNA along with

DNA was purified to enable detection of RNA viruses such as those belonging to the Flavivirus genus with one example being Powassan virus. Most extraction protocols only purify either DNA or RNA from the tick sample but not both. In order to maximize the nucleic acid in the tick both DNA and RNA are extracted in a single procedure.

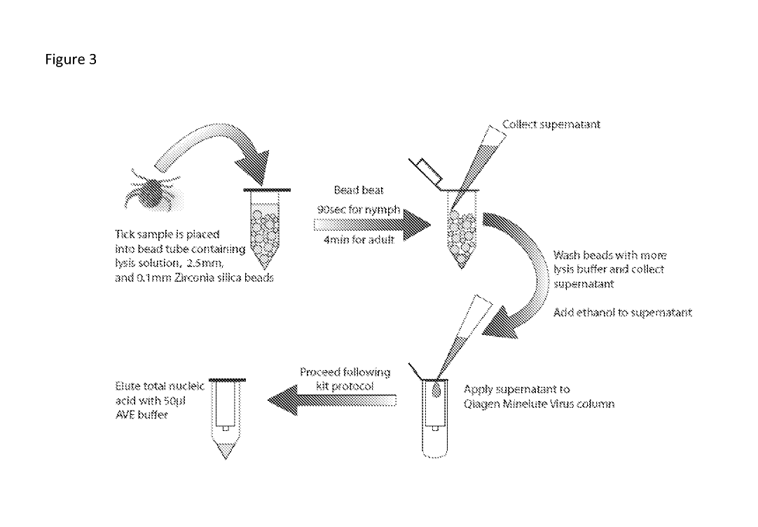

The tick sample is placed into a bead tube containing lysis solution, 2.5 mm, and 0.1 mm Zirconia silica beads. The sample is bead beat for 90 sec for nymph or 4 min for adult ticks. The beads were then washed with more lysis buffer and the supernatant was collected. Ethanol was added to the supernatant. The supernatant was then applied to a Qiagen Minelute Virus column and purified using the kit protocol. Total nucleic acid was eluted with 50 μl AVE buffer. FIG. 3 shows tick extraction of adult Dermacentor variabilis.

### Detection and Identification of Known and Potential Tick Pathogens

TABLE 3

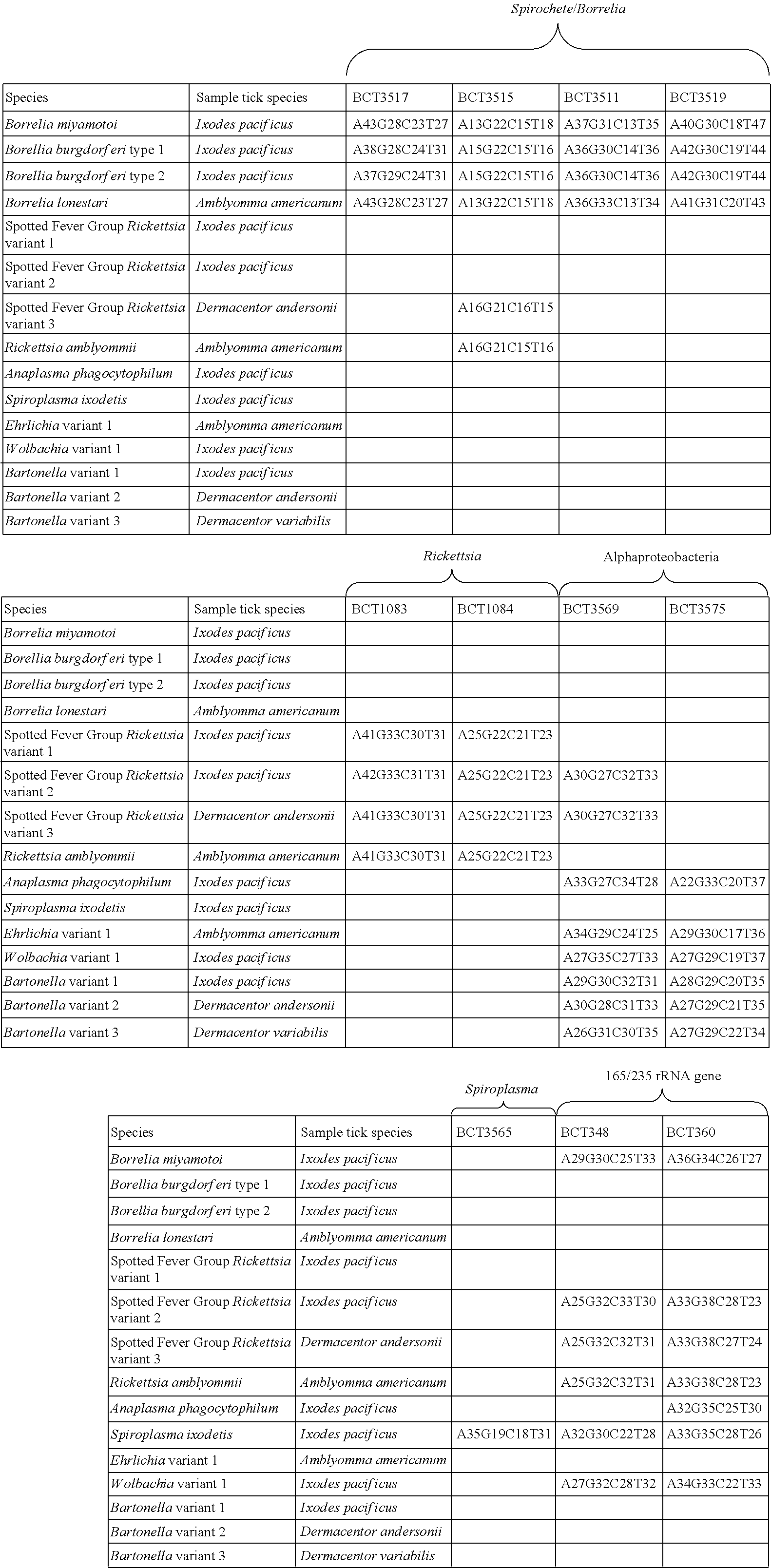

The Tick Borne Surveillance assay detected and identified known human pathogens from ticks. Additionally, it has the ability to identify novel pathogens that have yet to be characterized. These punative pathogens belong to known disease groups such as the Spotted Fever Rickettsia, but possess slightly different base count signatures from the known pathogens.

### Detection of Tick Endosymbionts

The Tick Borne Surveillance assay detected the natural endosymbiont bacteria in various species of ticks (Table 4). Tick endosymbionts were distinguished from pathogen candidates by the fact they were found in the majority of the ticks for a given species including farm raised ticks and field collected samples and in various tick life stages.

The endosymbionts were found to be specific to a given species of tick and allow for identification of the tick species.

TABLE 4

| | Endosymbiont | | | | | |
|---|---|---|---|---|---|---|
| Tick Species | Genus | Stage | Organism | BCT3515 | BCT1083 | BCT3569 |
| Ixodes scapularis | Rickettsia | Adult | Rickettsia variant 6 | A17G21C16T14 | A42G32C30T31 | A30G27C32T33 |
| | | Egg Mass | Rickettsia variant 6 | A17G21C16T14 | A42G32C30T31 | A30G27C32T33 |
| Dennacentor occidentalis | Francisella | Adult | Francisella varaiant 1 | Not Expected | Not Expected | Not Expected |
| Dennacentor variabilis | Francisella | Adult | Francisella varaiant 2 | Not Expected | Not Expected | Not Expected |
| Dennacentor andersoni | Francisella | Adult | Francisella varaiant 1 | Not Expected | Not Expected | Not Expected |

| Tick Species | BCT3575 | BCT2328 | BCT2332 | BCT348 | BCT360 |
|---|---|---|---|---|---|
| Ixodes scapularis | Not Expected | Not Expected | Not Expected | A26G31C32T31 | A34G37C27T24 |
| | Not Expected | Not Expected | Not Expected | A26G31C32T31 | A34G37C27T24 |
| Dennacentor occidentalis | A28G31C18T35 | A17G23C10T32 | A31G22C16T32 | A24G32C28T32 | A32G36C27T27 |
| Dennacentor variabilis | A27G32C18T35 | A17G23C09T33 | A31G22C16T32 | A24G32C28T32 | A32G36C27T27 |
| Dennacentor andersoni | A28G31C18T35 | A17G23C10T32 | A31G22C16T32 | A24G32C28T32 | A32G36C27T27 |

### Detection of Co-infection in a Single Tick and Detection of Multiple Pathogens

TABLE 5

| | BCT3517 | | | |
|---|---|---|---|---|
| Result | (flagellin) | BCT3515 (rplB) | BCT3569 (gltA) | BCT3575 (rpoB) |
| Borrelia burgdorferi | A37G29C24T31 | A15G22C15T16 | Not Expected | Not Expected |
| Anaplasma phagocytophilum | Not Expected | Not Expected | A33G27C34T28 | A22G33C20T37 |

| | BCT360 | | |
|---|---|---|---|
| Result | (23S rDNA) | BCT3511 (gyrB) | BCT3519 (hbb) |
| Borrelia burgdorferi | No Detection | A36G30C14T36 | A42G30C19T44 |
| Anaplasma phagocytophilum | A32G35C25T30 | Not Expected | Not Expected |

A co-infection of two known human pathogens was detected from a single I. pacificus tick (Table 5). The broad-range aspect of the Tick Borne Surveillance assay allows for detection of co-infections that are present even when the infecting organisms are unknown and in a single assay.

Detection of Multiple Borrelia Species in a Single Extraction of 4 Adult I. pacificus Ticks

TABLE 6

| | BCT3517 | | | |
|---|---|---|---|---|
| Result | (flagellin) | BCT3515 (rplB) | BCT3511 (gyrB) | BCT3519 (hbb) |
| Borrelia miyamotoi | A43G28C23T27 | No Detection | A37G31C13T35 | A40G30C18T47 |
| Borrelia burgdorferi | A38G28C24T31 | A15G22C15T16 | A36G30C14T36 | No Detection |

The assay was able to detect, identify, and differentiate between different species of Borrelia in the same sample (Table 6). The mixture of two Borrelia species was identified in a single assay. This has an advantage over qPCR with probes since while qPCR can detect mixtures, it can only do with probes for specific targets and if the targets are known in advance. This assay also shows an advantage over identification by sequencing since when a mixture is present it does not require cloning and sequencing of the PCR product to detect both species.

In conclusion, the bacteria and endosymbionts of field collected ticks from California were analyzed and compared to farm raised ticks of the same species from Oklahoma.

The distribution and prevalence of the vector-borne pathogens were mapped based on counties and compared to reported disease prevalence.

The study showed detection of both pathogenic bacteria and tick endosymbionts with resolution down to the species level for many of the disease causing organisms such as Francisella, Borrelia, Rickettsia, Ehrlichia, Bartonella, Anaplasma, Coxiella, Spiroplasma, and Flavivirusess. The extraction method allowed for the recovery of both DNA and RNA from a single tick or pool of ticks. The Tick Borne Surveillance assay allowed for the detection of Borrelia burgdorferi from a single infected nymph.

The assay detected co-infections of different pathogens in a single tick. The assay can detect and differentiate between different species of Borrelia in the same sample. The assay also detects endosymbionts in different tick species at various life stages.

### Example 2

### De Novo Determination of Base Composition of Amplicons Using Molecular Mass Modified Deoxynucleotide Triphosphates

Because the molecular masses of the four natural nucleobases have a relatively narrow molecular mass range (A=313.058, G=329.052, C=289.046, T=304.046, values in Daltons—See, Table 7), a persistent source of ambiguity in assignment of base composition may occur as follows: two nucleic acid strands having different base composition may have a difference of about 1 Da when the base composition difference between the two strands is G [symbol] A (−15.994) combined with C [symbol] T (+15.000). For example, one 99-mer nucleic acid strand having a base composition of A₂₇G₃₀C₂₁T₂₁ has a theoretical molecular mass of 30779.058 while another 99-mer nucleic acid strand having a base composition of A₂₆G₃₁C₂₂T₂₀ has a theoretical molecular mass of 30780.052 is a molecular mass difference of only 0.994 Da. A 1 Da difference in molecular mass may be within the experimental error of a molecular mass measurement and thus, the relatively narrow molecular mass range of the four natural nucleobases imposes an uncertainty factor in this type of situation. One method for removing this theoretical 1 Da uncertainty factor uses amplification of a nucleic acid with one mass-tagged nucleobase and three natural nucleobases.

Addition of significant mass to one of the 4 nucleobases (dNTPs) in an amplification reaction, or in the primers themselves, will result in a significant difference in mass of the resulting amplicon (greater than 1 Da) arising from ambiguities such as the G [symbol] A combined with C [symbol] T event (Table 7). Thus, the same G [symbol] A (−15.994) event combined with 5-Iodo-C [symbol] T (−110.900) event would result in a molecular mass difference of 126.894 Da. The molecular mass of the base composition A₂₇G₃₀5-Iodo-C₂₁T₂₁ (33422.958) compared with A₂₆G₃₁5-Iodo-C₂₂T₂₀, (33549.852) provides a theoretical molecular mass difference is +126.894. The experimental error of a molecular mass measurement is not significant with regard to this molecular mass difference. Furthermore, the only base composition consistent with a measured molecular mass of the 99-mer nucleic acid is A₂₇G₃₀5-Iodo-C₂₁T₂₁. In contrast, the analogous amplification without the mass tag has 18 possible base compositions.

TABLE 7
Molecular Masses of Natural Nucleobases and the
Mass-Modified Nucleobase 5-Iodo-C and Molecular
Mass Differences Resulting from Transitions

| Nucleobase | Molecular Mass | Transition | Δ Molecular Mass |
|---|---|---|---|
| A | 313.058 | A-->T | −9.012 |
| A | 313.058 | A-->C | −24.012 |
| A | 313.058 | A-->5-Iodo-C | 101.888 |
| A | 313.058 | A-->G | 15.994 |
| T | 304.046 | T-->A | 9.012 |
| T | 304.046 | T-->C | −15.000 |
| T | 304.046 | T-->5-Iodo-C | 110.900 |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5-Iodo-C | 85.894 |

Mass spectra of bioagent-identifying amplicons may be analyzed using a maximum-likelihood processor, such as is widely used in radar signal processing. This processor first makes maximum likelihood estimates of the input to the mass spectrometer for each primer by running matched filters for each base composition aggregate on the input data. This includes the response to a calibrant for each primer.

The algorithm emphasizes performance predictions culminating in probability-of-detection versus probability-of-false-alarm plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bacterial and viral bioagents and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent-identifying amplicons for each primer are calibrated and a final maximum likelihood amplitude estimate per organism is made based upon the multiple single primer estimates. Models of all system noise are factored into this two-stage maximum likelihood calculation. The processor reports the number of molecules of each base composition contained in the spectra. The quantity of amplicon corresponding to the appropriate primer set is reported as well as the quantities of primers remaining upon completion of the amplification reaction.

Base count blurring may be carried out as follows. Electronic PCR can be conducted on nucleotide sequences of the desired bioagents to obtain the different expected base counts that could be obtained for each primer pair. See for example, Schuler, Genome Res. 7:541-50, 1997; or the e-PCR program available from National Center for Biotechnology Information (NCBI, NIH, Bethesda, Md.). In one embodiment one or more spreadsheets from a workbook comprising a plurality of spreadsheets may be used (e.g., Microsoft Excel). First, in this example, there is a worksheet with a name similar to the workbook name; this worksheet contains the raw electronic PCR data. Second, there is a worksheet named “filtered bioagents base count” that contains bioagent name and base count; there is a separate record for each strain after removing sequences that are not identified with a genus and species and removing all sequences for bioagents with less than 10 strains. Third, there is a worksheet, “Sheet1” that contains the frequency of substitutions, insertions, or deletions for this primer pair. This data is generated by first creating a pivot table from the data in the “filtered bioagents base count” worksheet and then executing an Excel VBA macro. The macro creates a table of differences in base counts for bioagents of the same species, but different strains. One of ordinary skill in the art understands the additional pathways for obtaining similar table differences without undo experimentation.

Application of an exemplary script, involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by taking the most abundant strain's base type composition and adding it to the reference set and then the next most abundant strain's base type composition is added until the threshold is met or exceeded. The current set of data was obtained using a threshold of 55%, which was obtained empirically.

For each base count not included in the reference base count set for that bioagent, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules that aim to minimize the number of changes and, in instances with the same number of changes, minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as one, two, or more substitutions, one, two, or more insertions, one, two, or more deletions, and combinations of substitutions and insertions or deletions. The different classes of nucleobase changes and their probabilities of occurrence have been delineated in U.S. Patent Application Publication No. 2004209260 (U.S. application Ser. No. 10/418,514) which is incorporated herein by reference in entirety.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, internet web sites, and the like) cited in the present application is incorporated herein by reference in its entirety.

1. A composition, comprising at least one purified oligonucleotide primer pair that comprises forward and reverse primers, wherein said primer pair comprises nucleic acid sequences that are substantially complementary to nucleic acid sequences of two or more different tick-borne pathogens and/or or endosymbionts, wherein said primer pair is configured to produce amplicons comprising different base compositions that correspond to said two or more different bioagents.

2. The composition of claim 1, wherein said primer pair is configured to hybridize with conserved regions of said two or more different bioagents and flank variable regions of said two or more different bioagents.

3. The composition of claim 1, wherein said forward and reverse primers are about 15 to 35 nucleobases in length, and wherein the forward primer comprises at least 70%, at least 80%, at least 90%, at least 95%, or at least 100% sequence identity with a sequence selected from the group consisting of SEQ ID NOS: 1-18 and the reverse primer comprises at least 70% sequence identity with a sequence selected from the group consisting of SEQ ID NOS: 19-36.

4. The composition of claim 1, wherein said primer pair is selected from the group of primer pair sequences consisting of: SEQ ID NOS: 1:19, 2:20, 3:21, 4:22, 5:23, 6:24, 7:25, 8:26, 9:27, 10: 28; 11:29, 12:30, 13:31, 14:32, 15:33, 16:34, 17:35 and 18:36.

5. The composition of claim 1, wherein said forward and reverse primers are about 15 to 35 nucleobases in length, and wherein:
the forward primer comprises at least 70%, at least 80%, at least 90%, at least 95%, or at least 100% sequence identity with the sequence of SEQ ID NO: 1, and the reverse primer comprises at least 70%, at least 80%, at least 90%, at least 95%, or at least 100% sequence identity with the sequence of SEQ ID NO: 19;
the forward primer comprises at least 70%, at least 80%, at least 90%, at least 95%, or at least 100% sequence identity with the sequence of SEQ ID NO: 2, and the reverse primer comprises at least 70%, at least 80%, at least 90%, at least 95%, or at least 100% sequence identity with the sequence of SEQ ID NO: 20;
the forward primer comprises at least 70%, at least 80%, at least 90%, at least 95%, or at least 100% sequence identity with the sequence of SEQ ID NO: 3, and the reverse primer comprises at least 70%, at least 80%, at least 90%, at least 95%, or at least 100% sequence identity with the sequence of SEQ ID NO: 21;
the forward primer comprises at least 70%, at least 80%, at least 90%, at least 95%, or at least 100% sequence identity with the sequence of SEQ ID NO: 4, and the reverse primer comprises at least 70%, at least 80%, at least 90%, at least 95%, or at least 100% sequence identity with the sequence of SEQ ID NO: 22;
the forward primer comprises at least 70%, at least 80%, at least 90%, at least 95%, or at least 100% sequence identity with the sequence of SEQ ID NO: 5, and the reverse primer comprises at least 70%, at least 80%, at least 90%, at least 95%, or at least 100% sequence identity with the sequence of SEQ ID NO: 23;
the forward primer comprises at least 70%, at least 80%, at least 90%, at least 95%, or at least 100% sequence identity with the sequence of SEQ ID NO: 6, and the reverse primer comprises at least 70%, at least 80%, at least 90%, at least 95%, or at least 100% sequence identity with the sequence of SEQ ID NO: 24;
the forward primer comprises at least 70%, at least 80%, at least 90%, at least 95%, or at least 100% sequence identity with the sequence of SEQ ID NO: 7, and the reverse primer comprises at least 70%, at least 80%, at least 90%, at least 95%, or at least 100% sequence identity with the sequence of SEQ ID NO: 25;
the forward primer comprises at least 70%, at least 80%, at least 90%, at least 95%, or at least 100% sequence identity with the sequence of SEQ ID NO: 8, and the reverse primer comprises at least 70%, at least 80%, at least 90%, at least 95%, or at least 100% sequence identity with the sequence of SEQ ID NO: 26;
the forward primer comprises at least 70%, at least 80%, at least 90%, at least 95%, or at least 100% sequence identity with the sequence of SEQ ID NO: 9, and the reverse primer comprises at least 70%, at least 80%, at least 90%, at least 95%, or at least 100% sequence identity with the sequence of SEQ ID NO: 27;
the forward primer comprises at least 70%, at least 80%, at least 90%, at least 95%, or at least 100% sequence identity with the sequence of SEQ ID NO: 10, and the reverse primer comprises at least 70%, at least 80%, at least 90%, at least 95%, or at least 100% sequence identity with the sequence of SEQ ID NO: 28;
the forward primer comprises at least 70%, at least 80%, at least 90%, at least 95%, or at least 100% sequence identity with the sequence of SEQ ID NO: 11, and the reverse primer comprises at least 70%, at least 80%, at least 90%, at least 95%, or at least 100% sequence identity with the sequence of SEQ ID NO: 29;
the forward primer comprises at least 70%, at least 80%, at least 90%, at least 95%, or at least 100% sequence identity with the sequence of SEQ ID NO: 12, and the reverse primer comprises at least 70%, at least 80%, at least 90%, at least 95%, or at least 100% sequence identity with the sequence of SEQ ID NO: 30;
the forward primer comprises at least 70%, at least 80%, at least 90%, at least 95%, or at least 100% sequence identity with the sequence of SEQ ID NO: 13, and the reverse primer comprises at least 70%, at least 80%, at least 90%, at least 95%, or at least 100% sequence identity with the sequence of SEQ ID NO: 31;
the forward primer comprises at least 70%, at least 80%, at least 90%, at least 95%, or at least 100% sequence identity with the sequence of SEQ ID NO: 14, and the reverse primer comprises at least 70%, at least 80%, at least 90%, at least 95%, or at least 100% sequence identity with the sequence of SEQ ID NO: 32;
the forward primer comprises at least 70%, at least 80%, at least 90%, at least 95%, or at least 100% sequence identity with the sequence of SEQ ID NO: 15, and the reverse primer comprises at least 70%, at least 80%, at least 90%, at least 95%, or at least 100% sequence identity with the sequence of SEQ ID NO: 33;
the forward primer comprises at least 70%, at least 80%, at least 90%, at least 95%, or at least 100% sequence identity with the sequence of SEQ ID NO: 16, and the reverse primer comprises at least 70%, at least 80%, at least 90%, at least 95%, or at least 100% sequence identity with the sequence of SEQ ID NO: 34;
the forward primer comprises at least 70%, at least 80%, at least 90%, at least 95%, or at least 100% sequence identity with the sequence of SEQ ID NO: 17, and the reverse primer comprises at least 70%, at least 80%, at least 90%, at least 95%, or at least 100% sequence identity with the sequence of SEQ ID NO: 35; and/or the forward primer comprises at least 70%, at least 80%, at least 90%, at least 95%, or at least 100% sequence identity with the sequence of SEQ ID NO: 18, and the reverse primer comprises at least 70%, at least 80%, at least 90%, at least 95%, or at least 100% sequence identity with the sequence of SEQ ID NO: 36.

6. The composition of claim 1, wherein said different base compositions identify said two or more different bioagents at genus, species, or sub-species levels.

7. The composition of claim 1, wherein said two or more amplicons are 45 to 200 nucleobases in length.

8. A kit comprising the composition of claim 1.

9. The composition of claim 1, wherein said different bioagents are selected from the group consisting of: a Spirochete, an alphaproteobacteria, a Francisella sp., Coxiella burnetii, a Flavivirus, Borrelia miyamotoi, Borrelia burgdorferi type 1, Borrelia burgdorferi type 2, Borrelia lonestari, Spotted Fever Group Rickettsia variant 1, Spotted Fever Group Rickettsia variant 2, Spotted Fever Group Rickettsia variant 3, Rickettsia amblyommii, Anaplasma phagocytophilum, Spiroplasma ixodetis, Ehrlichia variant 1, Wolbachia variant 1, Bartonella variant 1, Bartonella variant 2, Bartonella variant 3, Francisella genus, or combinations thereof.

10. The composition of claim 1, wherein said primer pair is configured to hybridize with one or more nucleic acid sequences selected from the group consisting of: flagellin, rplB, gyrB, hbb, rnpA, gltA, rpoB, asd, gale, icd, IS1111A, S. kunkeii TraE-like gene, 16S rRNA gene, 23S rRNA gene and RdRp nucleic acids.

11. The composition of claim 1, wherein a non-templated T residue on the 5′-end of said forward and/or reverse primer is removed.

12. The composition of claim 1, wherein said forward and/or reverse primer further comprises a non-templated T residue on the 5′-end.

13. The composition of claim 1, wherein said forward and/or reverse primer comprises at least one molecular mass modifying tag.

14. The composition of claim 1, wherein said forward and/or reverse primer comprises at least one modified nucleobase.

15. The composition of claim 14, wherein said modified nucleobase is 5-propynyluracil or 5-propynylcytosine.

16. The composition of claim 14, wherein said modified nucleobase is a mass modified nucleobase.

17. The composition of claim 16, wherein said mass modified nucleobase is 5-Iodo-C.

18. The composition of claim 14, wherein said modified nucleobase is a universal nucleobase.

19. The composition of claim 18, wherein said universal nucleobase is inosine.

20. A kit, comprising at least one purified oligonucleotide primer pair that comprises forward and reverse primers that are about 20 to 35 nucleobases in length, and wherein said forward primer comprises at least 70%, at least 80%, at least 90%, at least 95%, or at least 100% sequence identity with a sequence selected from the group consisting of SEQ ID NOS: 1-18, and said reverse primer comprises at least 70% sequence identity with a sequence selected from the group consisting of SEQ ID NOS: 19-36.

21. A method of determining a presence of a tick-borne pathogen and/or or endosymbiont in at least one sample, the method comprising:
(a) amplifying one or more segments of at least one nucleic acid from said sample using at least one purified oligonucleotide primer pair that comprises forward and reverse primers that are about 20 to 35 nucleobases in length, and wherein said forward primer comprises at least 70%, at least 80%, at least 90%, at least 95%, or at least 100% sequence identity with a sequence selected from the group consisting of SEQ ID NOs: 1-18, and said reverse primer comprises at least 70% sequence identity with a sequence selected from the group consisting of SEQ ID NOs: 19-36 to produce at least one amplification product; and
(b) detecting said amplification product, thereby determining said presence of said tick-borne pathogen and/or or endosymbiont in said sample.

22. The method of claim 21, wherein (b) comprises detecting a molecular mass of said amplification product.

23. The method of claim 21, wherein (b) comprises determining a base composition of said amplification product, wherein said base composition identifies the number of A residues, C residues, T residues, G residues, U residues, analogs thereof and/or mass tag residues thereof in said amplification product, whereby said base composition indicates the presence of tick-borne pathogen and/or or endosymbiont in said sample or identifies said tick-borne pathogen and/or or endosymbiont in said sample.

24. The method of claim 23, comprising comparing said base composition of said amplification product to calculated or measured base compositions of amplification products of one or more known tick-borne pathogens and/or or endosymbionts present in a database with the proviso that sequencing of said amplification product is not used to indicate the presence of or to identify said tick-borne pathogen and/or or endosymbiont, wherein a match between said determined base composition and said calculated or measured base composition in said database indicates the presence of or identifies said tick-borne pathogen and/or or endosymbiont.

26. A system, comprising:
(a) a mass spectrometer configured to detect one or more molecular masses of amplicons produced using at least one purified oligonucleotide primer pair that comprises forward and reverse primers, wherein said primer pair comprises nucleic acid sequences that are substantially complementary to nucleic acid sequences of two or more different tick-borne pathogen and/or or endosymbiont bioagents; and
(b) a controller operably connected to said mass spectrometer, said controller configured to correlate said molecular masses of said amplicons with one or more tick-borne pathogen and/or or endosymbiont bioagent identities.

27. The system of claim 26, wherein said tick-borne pathogen and/or or endosymbiont bioagent identities are at genus, species, and/or sub-species levels.

28. The system of claim 27, wherein said forward and reverse primers are about 15 to 35 nucleobases in length, and wherein the forward primer comprises at least 70%, at least 80%, at least 90%, at least 95%, or at least 100% sequence identity with a sequence selected from the group consisting of SEQ ID NOS: 1-18, and the reverse primer comprises at least 70% sequence identity with a sequence selected from the group consisting of SEQ ID NOS: 19-36.

29. The system of claim 27, wherein said controller is configured to determine base compositions of said amplicons from said molecular masses of said amplicons, which base compositions correspond to said one or more tick-borne pathogen and/or or endosymbiont bioagent identities.

30. The system of claim 27, wherein said controller comprises or is operably connected to a database of known molecular masses and/or known base compositions of amplicons of known tick-borne pathogen and/or or endosymbiont bioagents produced with the primer pair.


SEQUENCE LISTING (36 sequences)

SEQ ID NO: 1 - DNA, 21 bp, Artificial Sequence
  Synthetic
tgctgaagagcttggaatgca

SEQ ID NO: 2 - DNA, 35 bp, Artificial Sequence
  Synthetic
tcgaataatgttattgagtttagatcttttggtac

SEQ ID NO: 3 - DNA, 24 bp, Artificial Sequence
  Synthetic
tgcatttgaaagcttggcattgcc

SEQ ID NO: 4 - DNA, 21 bp, Artificial Sequence
  Synthetic
tccacaaggtggtggtgaagg

SEQ ID NO: 5 - DNA, 22 bp, Artificial Sequence
  Synthetic
taagagcgcaccggtaagttgg

SEQ ID NO: 6 - DNA, 26 bp, Artificial Sequence
  Synthetic
tccaccaagagcaagatcaaataggc

SEQ ID NO: 7 - DNA, 25 bp, Artificial Sequence
  Synthetic
tgcatgcagatcatgaacaaaatgc

SEQ ID NO: 8 - DNA, 31 bp, Artificial Sequence
  Synthetic
tgcatcacttggttgatgataagatacatgc

SEQ ID NO: 9 - DNA, 35 bp, Artificial Sequence
  Synthetic
tgagggttttatgcttaaagttggttttattggtt

SEQ ID NO: 10 - DNA, 30 bp, Artificial Sequence
  Synthetic
tcagctagaccttttaggtaaagctaagct

SEQ ID NO: 11 - DNA, 23 bp, Artificial Sequence
  Synthetic
tcgccgtggaaaaatcctacgct

SEQ ID NO: 12 - DNA, 26 bp, Artificial Sequence
  Synthetic
tcagtatgtatccaccgtagccagtc

SEQ ID NO: 13 - DNA, 34 bp, Artificial Sequence
  Synthetic
tgtatatgttgaaactatctcacatggttgatga

SEQ ID NO: 14 - DNA, 24 bp, Artificial Sequence
  Synthetic
tgcttttattgttggccagatggg

SEQ ID NO: 15 - DNA, 23 bp, Artificial Sequence
  Synthetic
tttcgatgcaacgcgaagaacct

SEQ ID NO: 16 - DNA, 23 bp, Artificial Sequence
  Synthetic
tctgttcttagtacgagaggacc

SEQ ID NO: 17 - DNA, 25 bp, Artificial Sequence
  Synthetic
tagccgagccatctggtacatgtgg

SEQ ID NO: 18 - DNA, 29 bp, Artificial Sequence
  Synthetic
tgtgtctacaacatgatgggaaagagaga

SEQ ID NO: 19 - DNA, 27 bp, Artificial Sequence
  Synthetic
tacagcaattgcttcatcttgatttgc

SEQ ID NO: 20 - DNA, 27 bp, Artificial Sequence
  Synthetic
tggacgaaaatacgcaacatgatgatc

SEQ ID NO: 21 - DNA, 28 bp, Artificial Sequence
  Synthetic
tcattttagcacttcctccagcagaatc

SEQ ID NO: 22 - DNA, 18 bp, Artificial Sequence
  Synthetic
tcggctgtccccaaggag

SEQ ID NO: 23 - DNA, 24 bp, Artificial Sequence
  Synthetic
tcaagcgatctacccgcattacaa

SEQ ID NO: 24 - DNA, 24 bp, Artificial Sequence
  Synthetic
tcaagcgatctacccgcattacaa

SEQ ID NO: 25 - DNA, 18 bp, Artificial Sequence
  Synthetic
tccatgtgctggtcccca

SEQ ID NO: 26 - DNA, 23 bp, Artificial Sequence
  Synthetic
tcaccaaaacgctgaccaccaaa

SEQ ID NO: 27 - DNA, 31 bp, Artificial Sequence
  Synthetic
tgattcgatcatacgagacattaaaactgag

SEQ ID NO: 28 - DNA, 31 bp, Artificial Sequence
  Synthetic
tctcacctacagctttaaagccagcaaaatg

SEQ ID NO: 29 - DNA, 24 bp, Artificial Sequence
  Synthetic
tagccttttctccggcgtagatct

SEQ ID NO: 30 - DNA, 27 bp, Artificial Sequence
  Synthetic
taaacgtccgataccaatggttcgctc

SEQ ID NO: 31 - DNA, 26 bp, Artificial Sequence
  Synthetic
tgtgcttttcctccaaatggttgttg

SEQ ID NO: 32 - DNA, 25 bp, Artificial Sequence
  Synthetic
tggcaaaactaccatattcgcgttg

SEQ ID NO: 33 - DNA, 21 bp, Artificial Sequence
  Synthetic
tacgagctgacgacagccatg

SEQ ID NO: 34 - DNA, 22 bp, Artificial Sequence
  Synthetic
tttcgtgcttagatgctttcag

SEQ ID NO: 35 - DNA, 26 bp, Artificial Sequence
  Synthetic
tctctggaaagccagtggtcttcatt

SEQ ID NO: 36 - DNA, 21 bp, Artificial Sequence
  Synthetic
tgctcccagccacatgtacca